(19) United States
(12) Patent Application Publication
(10) Pub. No.: US 2024/0216548 A1
(43) Pub. Date: Jul. 4, 2024

(54) METHOD FOR PREPARING THERAGNOSTIC CARBON QUANTUM-DOT NANOMEDICINES FOR TARGETED DRUG DELIVERY TO ACUTE-MYELOID LEUKEMIA

(71) Applicants: Madeeha Shehzad Lodhi, Lahore (PK); Muhammad Tahir Khan, Lahore (PK); Shabbir Muhammad, Abha (SA); H. Algarni, Abha (SA); Tayyaba Yasin, Lahore (PK); Dong Qing Wei, Shanghai (CN); Sharifah Mohammed Ali Al-Moayid, Abha (SA)

(72) Inventors: Madeeha Shehzad Lodhi, Lahore (PK); Muhammad Tahir Khan, Lahore (PK); Shabbir Muhammad, Abha (SA); H. Algarni, Abha (SA); Tayyaba Yasin, Lahore (PK); Dong Qing Wei, Shanghai (CN); Sharifah Mohammed Ali Al-Moayid, Abha (SA)

(21) Appl. No.: 18/425,302

(22) Filed: Jan. 29, 2024

(51) Int. Cl. A61K 49/00; A61K 36/58; A61K 47/12; A61K 47/18; A61K 47/69

(52) U.S. Cl. CPC A61K 49/0067; A61K 36/58; A61K 47/12; A61K 47/183; A61K 47/6929; A61K 49/0058; A61K 2236/33; A61K 2236/51

(57) ABSTRACT

The method for preparing theragnostic carbon quantum dot nanomedicine composition comprises collecting and processing fresh plant samples of Azadirachta indica; mixing 50 g of crushed dried neem leaves powder with 500 mL of methanol and left to extract for 1 week; filtering the mixture and condensing using a rotary evaporator, and lyophilizing to obtain the extract; dissolving 0.57 g L-cysteine in 10 mL of deionized water and mixing with 1.5 g of citric acid to form a solution; ultrasonicating the solution for 10 minutes and then heating in a microwave oven for 4 minutes; cooling the solution to room temperature and then dissolving in 10 mL of deionized water to obtain a brown solution; centrifuging the brown solution at 9000 rpm for 10 minutes to remove impurities and filtering with a filter; and extracting the Carbon quantum dot (CQD) solution with ethyl acetate and storing in a refrigerator at 4° C.

### FIELD OF THE INVENTION

The present disclosure relates to the field of nanomedicine and specifically pertains to a method for preparing theragnostic carbon quantum-dot nanomedicines designed for targeted drug delivery to acute myeloid leukemia (AML). The invention encompasses a novel approach that integrates the unique properties of carbon quantum dots with therapeutic and diagnostic capabilities to enhance the precision and effectiveness of drug delivery for the treatment of AML. The method involves the conjugation of specific biomolecules to carbon quantum dots, allowing for targeted delivery to AML cells while enabling simultaneous diagnostic imaging, thus offering a theragnostic solution for the treatment and monitoring of acute myeloid leukemia.

### BACKGROUND OF THE INVENTION

Cancer has been a significant health issue for decades, and with increasing rates of cancer diagnosis, there is a need for more effective and targeted cancer treatments. One approach to improving cancer treatment is using nanomedicines designed to target cancer cells and deliver drugs to them precisely. Carbon quantum dot (CQD) nanomedicines are gaining popularity due to their unique properties, including biocompatibility, stability, and high drug loading capacity. This study will discuss the development of theragnostic CQD nanomedicines, specifically those based on the natural product A. indica and CD33 antibodies, for targeted drug delivery to acute myeloid leukemia (AML).

AML is a type of blood cancer that affects the bone marrow and blood cells. It is a highly aggressive and lethal disease, with a five-year survival rate of only 27%. It is characterized by the rapid growth and accumulation of abnormal white blood cells, which can interfere with the body's normal functioning. AML is typically treated with chemotherapy, but the side effects of this treatment can be severe, and there is a need for more targeted and effective treatments.

CQDs are nanomaterials that have been extensively studied for their use in drug delivery and imaging applications. They are typically less than 10 nm in diameter and are made from carbon-based materials such as graphene, carbon nanotubes, and carbon black. CQDs have several unique properties which make them ideal for use in theragnostic nanomedicines. Additionally, CQDs can be functionalized with targeting ligands, such as antibodies, to target cancer cells specifically.

CQDs have been extensively studied for their use in drug delivery and imaging applications in cancer therapy. Their small size allows for easy entry into cells, making them ideal for targeted drug delivery. CQDs can be loaded with various drugs, including chemotherapy agents and siRNA, and can be conjugated with targeting ligands, such as antibodies, to target cancer cells specifically.

A. indica, also known as neem, is a natural product used for centuries in traditional medicine. It has been shown to have a wide range of biological activities, including antifungal, antibacterial, antiviral, and anticancer properties. In recent years, neem has been studied extensively for its potential use in cancer treatment, and several neem-based products have been developed. Neem-based CQD nanomedicines have the potential to be highly effective cancer treatments due to the unique properties of neem and CQDs. Several studies have investigated using neem-based CQD nanomedicines for cancer treatment, including AML.

CD33 is a transmembrane protein expressed on the surface of myeloid cells, including AML cells. CD33 is a potential target for the development of antibody-based therapies for AML. CD33 is a potential target for developing antibody-based therapies for AML, and several CD33-targeted therapies have been developed, including gemtuzumab ozogamicin (GO). This CD33-targeted antibody-drug conjugate has been approved for the treatment of AML. One approach is to combine CD33-targeted antibodies with nanomedicines, such as CQDs. Developing targeted nanomedicines based on CQDs functionalized with CD33 antibodies represents a promising strategy for treating AML. CQDs are ideal for targeted drug delivery due to their small size, high biocompatibility, and excellent photoluminescence properties.

Theragnostics are a class of nano-sized medicine that combine diagnostic and therapeutic functions. They are designed to deliver drugs directly to cancer cells while providing real-time disease imaging. This enables doctors to monitor the progress of the treatment and adjust the dosage as needed. One approach to improving cancer treatment is using nanomedicines designed to target cancer cells and deliver drugs to them precisely. Theragnostic CQD nanomedicines are new nanomedicines combining therapeutic and diagnostic capabilities. These nanomedicines can be designed to specifically target cancer cells and deliver drugs to them while also providing imaging capabilities that can be used to monitor the disease's progression and the treatment's effectiveness. The development of CQD-based theragnostics has the potential to revolutionize AML treatment by enabling the targeted delivery of chemotherapy drugs to cancer cells.

In recent years, researchers have developed CQD-based theragnostic platforms that incorporate both chemotherapy drugs and imaging agents. These nanomedicines can selectively target cancer cells, leading to better treatment outcomes and reduced toxicity. Overall, the development of CQD-based nanomedicines for AML treatment represents a significant advancement in the field of cancer therapeutics, with the potential to improve patient outcomes and quality of life.

Drugs are smaller molecules and cannot trigger an immune response independently. Carrier proteins that are less immunogenic or non-immunogenic, and can trigger a humoral reaction to an immunogen, are necessary for delivering smaller peptides and medicines. The process develops developed five types of theragnostic nanomedicines. There is a lack of data on the characteristics and use of CQD as nanocarriers in nanoformulations.

In view of the foregoing discussion, it is portrayed that there is a need to have a method for preparing theragnostic carbon quantum dot nanomedicines for targeted drug delivery to acute myeloid leukemia.

### SUMMARY OF THE INVENTION

The present disclosure seeks to provide a method for preparing theragnostic carbon quantum dot nanomedicine composition and nanomedicines thereof for targeted drug delivery to acute myeloid leukemia. The fresh plant sample Azadirachta indica (A. indica) is collected from NIBGE Faisalabad, Pakistan. For the analysis of various phytoconstituents, different tests including alkaloids, flavonoids, tannins, terpenoids, steroids, and saponins are performed. CQDs is synthesized by chemical method with cysteine and citric acid and characterized by Fourier Transform Infrared (FTIR) Spectroscopy, UV-visible Spectroscopy, Energy Dispersive X-ray Spectroscopy (EDX), Dynamic Light Scattering (DLS), Scanning Electron Microscopy (SEM), and X-Ray Diffraction (XRD). The five theragnostic nanomedicines OH-CQD are prepared to investigate the effect of nanomedicines on targeted drug delivery. AML is induced by N-ethyl-N-nitrosourea (ENU) using intraperitoneal injection (IP). After four weeks, three rats from the negative control group are slaughtered and examined to diagnose AML. Then 24 rats of AML induction (ENU+NaCl) are used to evaluate the efficacy and drug binding to leukemia cells (in vivo and in vitro). 20 μl of the blood is separated for fluorescent analysis, and the remaining blood is processed for various biochemical tests. SPSS version 26 is used for data analysis, and p<0.05 is considered as significance. Phytochemical results revealed that the highest amounts of saponin, steroid, and terpenoid are in the aqueous extract of A. indica. Tannins are present in a moderate amount, and phenol, alkaloids, and flavonoids are present in a low amount. UV-vis spectroscopy exhibited an absorption peak of CQDs and conjugations of CQDs. The carbonyl, carboxyl, and hydroxyl groups have been confirmed through FTIR on the CQDs, conjugation of phytochemicals from methanolic extracts on these functional groups of CQDs and conjugated other targeted nanoformulations with CD33 protein. XRD results showed that the presence of graphitic carbon, a distinct diffraction peak appears in the range of 2=19-24°, corresponding to the plane of (002). Although the zeta potential of CQDs and targeted nanoformulations confirmed the presence of negatively charged moieties on the surface of CQDs. DLS observed monodispersed and a narrow size range with 2-5 nm, except for CQDs attached phytochemicals, which showed a little aggregation tendency that is overcome by protein conjugation and made particles stable. SEM results revealed that the CQDs, cytarabine-conjugated GNPs and both targeted nanoformulations are irregular spherical structure and around 30-40 nm in diameter. The EDX spectrum of the CQDs reveals peaks corresponding to O, C, Na, Rb, Au, S, and Pb. The mean WBCs level of the treatment groups is significantly near to the control (p<0.047). The effective nanomedicines are Aldehyde-CQD, standard drug nanoformulation, extract, NF1 and NF2 groups than others treatment groups. These results evaluate the efficacy of theranostic nanomedicines against AML and proved that these nanomedicines have potential to deliver anti-AML drug and CD33 protein can be used as ligand.

In an embodiment, a theragnostic carbon quantum dot nanomedicines composition for targeted drug delivery to acute myeloid leukemia is disclosed. The composition comprises a powder extract of Azadirachta indica, from 40-60 grams in 400-600 mL of methanol; a powder extract of L-cysteine, from 0.3-1 grams in 5-15 mL of deionized water; an aqueous extract of citric acid, from 1-2 grams; and an aqueous extract of ethyl acetate, from 0-10 mL.

In an embodiment, a method for preparing theragnostic carbon quantum dot nanomedicines for targeted drug delivery to acute myeloid leukemia is disclosed. The method includes collecting and processing fresh plant samples of Azadirachta indica.

The method further includes mixing 50 g of crushed dried neem leaves powder with 500 mL of methanol and left to extract for 1 week.

The method further includes filtering the mixture and condensing using a rotary evaporator, and lyophilizing to obtain the extract.

The method further includes dissolving 0.57 g L-cysteine in 10 mL of deionized water and mixing with 1.5 g of citric acid to form a solution.

The method further includes ultrasonicating the solution for 10 minutes and then heating in a microwave oven for 4 minutes.

The method further includes cooling the solution to room temperature and then dissolving in 10 mL of deionized water to obtain a brown solution.

The method further includes centrifuging the brown solution at 9000 rpm for 10 minutes to remove impurities and filtering with a filter.

The method further includes extracting the Carbon quantum dot (CQD) solution with ethyl acetate and storing in a refrigerator at 4° C.

An object of the present disclosure is to provide a method for preparing theragnostic carbon quantum-dot nanomedicines that exhibit enhanced targeting capabilities specifically tailored for acute myeloid leukemia (AML) cells.

Another object of the present disclosure is to achieve precise and controlled drug delivery to AML cells, minimizing off-target effects and improving the therapeutic efficacy of the administered drugs.

Another object of the present disclosure is to integrate theragnostic features, allowing simultaneous therapeutic drug delivery and diagnostic imaging. This dual functionality enables real-time monitoring of the treatment response and enhances the overall therapeutic outcome.

Another object of the present disclosure is to harness the unique properties of carbon quantum dots, including their biocompatibility and fluorescence, to develop nanomedicines capable of targeted drug delivery and imaging for AML.

Another object of the present disclosure is to enhance the bioavailability of therapeutic agents by utilizing carbon quantum dots as carriers, promoting improved solubility and stability of the drugs during delivery to AML cells.

Another object of the present disclosure is to minimize side effects associated with conventional chemotherapy by selectively delivering drugs to AML cells, thereby sparing healthy tissues from unnecessary exposure to cytotoxic agents.

Another object of the present disclosure is to contribute to the development of personalized treatment approaches for AML, allowing customization of therapeutic interventions based on the specific characteristics of individual patients.

Another object of the present disclosure is to optimize the conjugation strategies between carbon quantum dots and biomolecules, ensuring efficient drug loading, stability, and sustained release at the targeted AML sites.

Another object of the present disclosure is to provide theragnostic nanomedicines with long-term storage stability, allowing for convenient and practical use in clinical settings.

Another object of the present disclosure is to facilitate the clinical translation of theragnostic carbon quantum-dot nanomedicines, offering a novel and effective approach for the targeted treatment of acute myeloid leukemia.

Yet another object of the present invention is to deliver an expeditious and cost-effective method for preparing theragnostic carbon quantum dot nanomedicines for targeted drug delivery to acute myeloid leukemia.

To further clarify the advantages and features of the present disclosure, a more particular description of the invention will be rendered by reference to specific embodiments thereof, which are illustrated in the appended drawings. It is appreciated that these drawings depict only typical embodiments of the invention and are therefore not to be considered limiting of its scope. The invention will be described and explained with additional specificity and detail in the accompanying drawings.

### BRIEF DESCRIPTION OF FIGURES

These and other features, aspects, and advantages of the present disclosure will become better understood when the following detailed description is read concerning the accompanying drawings in which like characters represent like parts throughout the drawings, wherein:

FIG. 1 illustrates a flow chart of a method for preparing theragnostic carbon quantum dot nanomedicines for targeted drug delivery to acute myeloid leukemia in accordance with an embodiment of the present disclosure;

FIG. 2 illustrates a schematic presentation of the synthesis of CQD;

FIG. 3 illustrates a proposed model of a reaction sequence for the synthesis of CQD theragnostic nanoformulations;

FIG. 4 illustrates the UV-Vis absorbance spectra of CQD (a). UV-Vis absorbance spectra of standard drug NF (b). UV-Vis absorbance spectra of OH-CQD (c) and NF1 (d). UV-Vis absorbance spectra of aldehyde-CQD (e) and NF2 (f);

FIG. 5 illustrates the FTIR spectra of CQDs nanoparticles (a). FTIR spectra of standard drug NF (b). FTIR spectra of OH-CQDs (c) and NF1 (d). FTIR spectra of aldehyde-CQD (e) and NF2 (f);

FIG. 6 illustrates the XRD of CQD (a). XRD of standard drug NF (b). XRD of NF1 (c) and NF2 (d);

FIG. 7 illustrates the Zeta potential of CQD nanoparticles (a). Zeta potential of standard drug NF (b). Zeta potential of targeted NF1 (c). Zeta potential of targeted NF2 (d);

FIG. 8 illustrates the DLS analysis of CQD nanoparticles (a). DLS analysis of standard drug NF (b). DLS analysis of OH-CQD (c) and NF1 (d). DLS analysis of aldehyde-CQD (e) and NF2 (f);

FIG. 9 illustrates a) SEM images of CQDs at 200 nm and 100 nm scales, b) SEM images of standard drug NF at 200 nm and 100 nm scales, c) SEM images of targeted NF1 at 300 nm and 100 nm scales, and d) SEM images of targeted NF2 at 300 nm and 100 nm scales;

FIG. 10 illustrates a) EDX image of elemental composition of the CQDs and b) EDX micrograph of CQDs;

FIG. 11 illustrates the schematic presentation of injected theragnostic nanoformulations group in rats;

FIG. 12 illustrates the weight of rats (Induction) from Day-1 till Day-25;

FIG. 13 illustrates the Doses of rats (Induction) from Day-1 to till Day-23;

FIG. 14 illustrates the weight of rats (Therapeutic) from Day 1 till Day 12;

FIG. 15 illustrates In vitro receptor binding of CQD theragnostic nanomedicine on leukemia blood slides;

FIG. 16 illustrates the complete blood count levels in control and treatment groups;

FIG. 17 illustrates the LFT Parameters in control and treatment groups;

FIG. 18 illustrates the RFT Profile in control and treatment groups;

FIG. 19 illustrates the Macroscopic examination of rat blood treated with theragnostic nanoformulations. (a) Control group (b) Negative control (c) Nanocarrier (d) Extract based group (e) Standard drug nanoformulation (f) OH-CQD group (g) Aldehyde-CQD (h) NF1 (i) NF2;

FIG. 20 illustrates a Table depicting labeled treated groups of theragnostic nanomedicines.

FIG. 21 illustrates a Table depicting the phytochemical content of neem plant (A. india).

FIG. 22 illustrates a Table depicting the elemental composition of the CQDs.

Further, skilled artisans will appreciate those elements in the drawings are illustrated for simplicity and may not have necessarily been drawn to scale. For example, the flow charts illustrate the method in terms of the most prominent steps involved to help to improve understanding of aspects of the present disclosure. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having the benefit of the description herein.

### DETAILED DESCRIPTION

To promote an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

Reference throughout this specification to “an aspect”, “another aspect” or similar language means that a particular feature, structure, or characteristic described in connection with the embodiment is included in at least one embodiment of the present disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this invention belongs. The system, methods, and examples provided herein are illustrative only and not intended to be limiting.

Embodiments of the present disclosure will be described below in detail concerning the accompanying drawings.

In an embodiment, a theragnostic carbon quantum dot nanomedicines composition for targeted drug delivery to acute myeloid leukemia is disclosed. The composition comprises a powder extract of Azadirachta indica, from 40-60 grams in 400-600 mL of methanol; a powder extract of L-cysteine, from 0.3-1 grams in 5-15 mL of deionized water; an aqueous extract of citric acid, from 1-2 grams; and an aqueous extract of ethyl acetate, from 0-10 mL.

In another embodiment, the weight amount of Azadirachta indica, L-cysteine, citric acid, methanol, and deionized water is 50 g, 0.57 g, 1.5 g, 500 mL, and 10 mL, respectively.

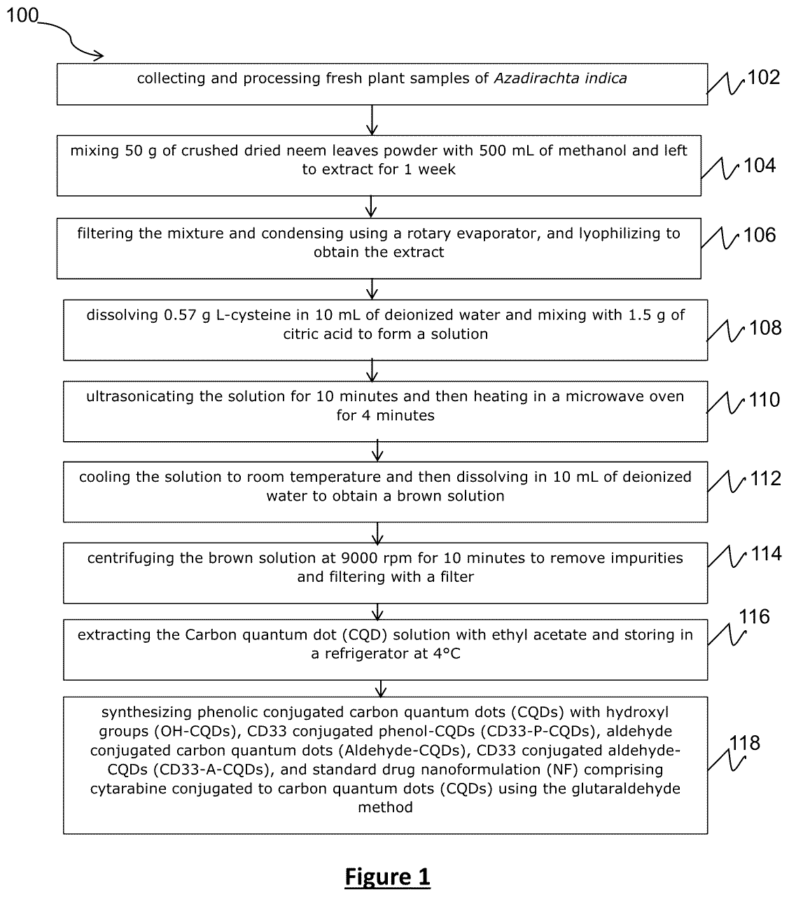

Referring to FIG. 1, a flow chart of a method for preparing theragnostic carbon quantum dot nanomedicines composition for targeted drug delivery to acute myeloid leukemia is illustrated in accordance with an embodiment of the present disclosure. At step 102, method 100 includes collecting and processing fresh plant samples of Azadirachta indica.

At step 104, method 100 includes mixing 50 g of crushed dried neem leaves powder with 500 mL of methanol and left to extract for 1 week.

At step 106, method 100 includes filtering the mixture and condensing using a rotary evaporator, and lyophilizing to obtain the extract.

At step 108, method 100 includes dissolving 0.57 g L-cysteine in 10 mL of deionized water and mixing with 1.5 g of citric acid to form a solution.

At step 110, method 100 includes ultrasonicating the solution for 10 minutes and then heating in a microwave oven for 4 minutes.

At step 112, method 100 includes cooling the solution to room temperature and then dissolving in 10 mL of deionized water to obtain a brown solution.

At step 114, method 100 includes centrifuging the brown solution at 9000 rpm for 10 minutes to remove impurities and filtering with a filter.

At step 116, method 100 includes extracting the Carbon quantum dot (CQD) solution with ethyl acetate and storing in a refrigerator at 4ºC.

At step 118, method 100 includes synthesizing phenolic conjugated carbon quantum dots (CQDs) with hydroxyl groups (OH-CQDs), CD33 conjugated phenol-CQDs (CD33-P-CQDs), aldehyde conjugated carbon quantum dots (Aldehyde-CQDs), CD33 conjugated aldehyde-CQDs (CD33-A-CQDs), and standard drug nanoformulation (NF) comprising cytarabine conjugated to carbon quantum dots (CQDs) using the glutaraldehyde method.

In another embodiment, synthesizing phenolic conjugated carbon quantum dots (CQDs) with hydroxyl groups (OH-CQDs), comprising the steps of conjugating particles with 4.28 μg/μl of CQDs and a hydroxyl group-containing phytochemical. Then, suspending lyophilized methanol extracts in a Tetrahydrofuran (THF) suspension containing N, N′-carbonyldiimidazole (CDI) at 50 mg/ml and mixing for 2 hours at room temperature. Then, washing activated particles with THE to remove excess CDI and byproducts. Then, resuspending the particles at 10 mg/ml in a cold coupling buffer and washing with ice-cold deionized water, wherein the cold coupling buffer containing 0.1M sodium carbonate at 9.5 pH. Thereafter, adding amine-containing nanoparticles (NPs) and allowing them to react by mixing for at least 18 hours at 4ºC.

In another embodiment, the particles are washed with ice-cold deionized water after resuspension.

In another embodiment, synthesizing CD33 conjugated phenol-CQDs (CD33-P-CQDs), comprising the steps of taking 10 mg of phenolic-conjugated CQDs (OH-CQDs) in 10 ml of coupling buffer adjusted to pH 8 with 1 M NaOH, wherein the coupling buffer comprises 50 mM sodium phosphate. Then, combining 50 μL of rabbit polyclonal CD33 in 10 ml of coupling buffer with the OH-CQDs solution, followed by thorough mixing. Then, adding 10 mg of 1-ethyl-3-[3-dimethylaminopropyl] carbodiimide (EDC) in a 1:1 ratio with CQDs to the solution to activate the carboxylic groups of the CQDs, while maintaining pH at 6.4 with 1 M HCl. Then, incubating a reaction mixture for 2 hours at 37° C. in a dark with continuous shaking at 100 rpm. Then, removing unattached molecules by centrifugation at 12,000 rpm for 10 minutes at 40° C., followed by washing three times with TBS, wherein 20 ml TBS is used for each time wash. Thereafter, suspending the washed particles in TBS buffer and storing them at −20° C. for later use.

In another embodiment, synthesizing aldehyde conjugated carbon quantum dots (Aldehyde-CQDs), comprising the steps of conjugating plant phytochemicals to amine-functionalized CQDs, wherein the plant phytochemicals are oxidized and suspended in 0.05M sodium carbonate and 0.1M sodium citrate at pH=9.6 and 1 mg mL 1. Then, adding amine-functionalized CQDs to the phytochemicals with an aldehyde group, dissolved in a buffer with a concentration of 1 to 10 mg/ml at pH 8-10, with a molar ratio of 1:4. Then, adding 10 μL of 5M-sodium cyanoborohydride in IN NaOH per mL of conjugation solution and allowing the solution to react for 2 hours on a rotator. Then, blocking unreacted sites by adding 10 μL ethanolamine, pH=9.6 per mL of the conjugation solution volume, and allowing it to react for 30 minutes on a rotator. Thereafter, purifying the particles through dialysis for 15 minutes and storing the purified particles at 4ºC.

In another embodiment, synthesizing CD33 conjugated aldehyde-CQDs (CD33-A-CQDs), comprising the steps of taking 10 mg of aldehyde-CQDs and 50 μL of CD33 in 10 ml of coupling buffer at pH 8 with 1 M NaOH, wherein the coupling buffer containing 50 mM sodium phosphate. Then, mixing the solutions thoroughly and adding 10 mg of 1-ethyl-3-[3-dimethylaminopropyl] carbodiimide (EDC) in a 1:1 ratio with CQDs to activate the carboxylic groups at pH 6.4 with 1 M HCl. Then, incubating the reaction mixture for 2 hours at 37 ºC in the dark with continuous shaking at 100 rpm. Then, centrifuging the solution at 12,000 rpm for 10 minutes at 4ºC to remove unattached molecules and washing the particles three times with 20 ml each time TBS. Thereafter, suspending the washed particles in 1×TBS buffer and storing the suspended particles at −20° C.

In another embodiment, preparing a standard drug nanoformulation (NF) comprising cytarabine conjugated to carbon quantum dots (CQDs) using the glutaraldehyde method, comprising the steps of dispersing 100 mg of CQDs in 100 ml of coupling buffer through sonication for 10 minutes at high power, wherein the buffer has 0.01 M pyridine HCl, pH: 6.00, 0.1 M NaCl. Then, centrifuging the CQD suspension at 14,000 rpm for 40 minutes and removing the supernatant after 10 minutes. Then, washing the CQDs with coupling buffer as described, and suspending them in a linking buffer, wherein the linking buffer contains coupling buffer with 5% glutaraldehyde. Then, shaking the CQDs in the linking buffer for 3 hours at 100 rpm at 25° C. and then centrifuging at 14,000 rpm for 20 minutes. Then, removing excess glutaraldehyde by aspirating the supernatant and washing the CQDs with coupling buffer as previously described. Then, dissolving 10 mg of cytarabine in 100 ml coupling buffer, reserving one ml as a pre-coupling buffer, and combining the remaining cytarabine solution with 100 mg of washed CQDs. Then, shaking the mixture from end to end for 24 hours at 25° C. and centrifuging the mixture at 8000 rpm at 4° C. to obtain standard drug nanoformulations (NFs). Then, keeping the characterized conjugated CQDs at −20° C. in a storage buffer solution of 0.01 M Tris-Cl, pH.

In another embodiment, the CQDs are dispersed in the coupling buffer through sonication for 10 minutes at high power.

The fresh plant sample of A. indica is collected from NIBGE Faisalabad in March 2022. The plants are found growing freely as wild plants and are identified by Dr. Yasin Ashraf, a botanist from the IMBB department. The 50 g of the crushed dried neem leaves powder is mixed with 500 mL of methanol and left to extract for 1 week. The mixture is then filtered, condensed using a rotary evaporator, and lyophilized to obtain the extract for further analysis.

In the next step, CQDs are prepared using a rapid microwave-assisted synthesis method. L-cysteine (0.57 g) is dissolved in 10 mL of deionized water and mixed with 1.5 g of citric acid. The solution is ultrasonicated for 10 minutes and then heated in a microwave oven for 4 minutes. After cooling the solution to room temperature, it is dissolved in 10 mL of deionized water to obtain a brown solution. The solution is then centrifuged at 9000 rpm for 10 minutes to remove impurities and filtered with a filter. The CQD solution is finally extracted with ethyl acetate and stored in a refrigerator at 4° C. until later use.

To characterize the CQDs, various techniques are employed, including UV-Vis spectroscopy, Fourier transform infrared (FTIR) spectroscopy, X-Ray diffraction (XRD), dynamic light scattering (DLS), Zeta potential, scanning electron microscopy (SEM), and energy dispersive X-ray (EDX). These techniques are used to determine the CQDs' morphology, chemical composition, and size.

The UV-Vis spectroscopy exhibited an absorption peak of CQDs at 364 nm. The FTIR analysis confirmed the presence of carbonyl, carboxyl, and hydroxyl functional groups on the CQD surface. The DLS results showed monodispersity and a narrow size range with 2-5 nm for CQDs. The SEM results revealed that the CQDs are irregular spherical structures around 30-40 nm in diameter. The EDX spectrum of the CQDs revealed peaks corresponding to O, C, Na, Rb, Au, S, and Pb.

The method prepared five theragnostic nanomedicines to investigate the effect of nanomedicines on targeted drug delivery. Due to their unique physical properties, the CQDs are used as the nanocarrier for all the nanomedicines. The process for making the theragnostic nanomedicines are discussed below.

### Phenolic Conjugated CQDs (OH-CQDs) (Non-Targeted):

The first nanomedicine involved CQDs-OH, which are further derivatives to provide different functional groups for biomolecule coupling. The particles are conjugated with 4.28 μg/μl of CQDs and a hydroxyl group-containing phytochemicals. Lyophilized methanol extracts are suspended in a Tetrahydrofuran (THF) suspension containing N, N′-carbonyldiimidazole (CDI) at 50 mg/ml and mixed for 2 hours at room temperature. The activated particles are washed with THE to remove excess CDI and byproducts before being resuspended at 10 mg/ml in a cold coupling buffer (0.1M sodium carbonate, pH=9.5) and washed with ice-cold deionized water. Finally, amine-containing NPs are added and allowed to react by mixing for at least 18 hours at 4ºC.

### CD33 Conjugated Phenol-CQDs (CD33-P-CQDs) (NF 1):

The NF1 linked phenolic-conjugated CQDs to rabbit polyclonal CD33 using the carbodiimide method (1-ethyl-3-[3-dimethylaminopropyl] carbodiimide cross-linking agent). 10 mg OH-CQDs are taken in 10 ml of coupling buffer (50 mM sodium phosphate) and set at pH 8 with 1 M NaOH. Then, 50 μL of rabbit polyclonal CD33 (500 μg total concentration) is taken in 10 ml of coupling buffer, and the two solutions are combined and mixed thoroughly. Next, 10 mg of EDC (1:1 of CQDs) is added to the solution to activate the carboxylic groups of the CQDs while maintaining the pH at 6.4 using 1 M HCl. The reaction mixture is then incubated for 2 hours at 37° C. in the dark with continuous shaking at 100 rpm. The remaining unattached molecules are removed by centrifugation at 12,000 rpm for 10 minutes at 40 C, followed by washing three times with TBS (20 ml each time). Finally, the washed particles are suspended in 1×TBS buffer and stored at −20° C. for later use.

### Aldehyde Conjugated CQDs (Aldehyde-CQDs) (Non-Targeted):

The aldehyde-CQDs involved conjugating plant phytochemicals to amine-functionalized CQDs. Oxidized phytochemicals are suspended in 0.05M sodium carbonate and 0.1M sodium citrate at pH=9.6 and 1 mg mL 1. Next, amine-functionalized CQDs are added to the phytochemicals that had an aldehyde group and are dissolved with a molar ratio of 1:4 in a buffer with a concentration of 1 to 10 mg/ml at pH 8-10. Then, 10 μL of 5M-sodium cyanoborohydride in IN NaOH per mL of conjugation solution is added, and the solution is allowed to react for 2 hours on a rotator. The unreacted sites are blocked by adding 10 μL ethanolamine, pH=9.6 per mL of the conjugation solution volume and allowed to react for 30 minutes on a rotator. The particles are then purified through dialysis for 15 minutes and stored at 4ºC.

### CD33 Conjugated Aldehyde-CQDs (CD33-A-CQDs) (NF 2):

The NF2 is prepared by taking 10 mg of aldehyde-CQDs and 50 μL of CD33 in 10 ml of coupling buffer (50 mM sodium phosphate) at pH 8 with 1 M NaOH. Both solutions are mixed thoroughly, and then 10 mg of EDC (1:1 of CQDs) is added to activate the carboxylic groups at pH 6.4 with 1 M HCl. The reaction mixture is incubated for 2 hours at 37° C. in the dark with continuous shaking at 100 rpm. The solution is then centrifuged at 12,000 rpm for 10 minutes at 40 C to remove any unattached molecules, followed by washing three times with TBS (20 ml each time). Finally, the washed particles are suspended in 1×TBS buffer and stored at −20° C. for later use.

### Standard Drug Nanoformulation:

The standard drug (cytarabine) is used and conjugated to CQDs using the glutaraldehyde method. First, 100 mg of CQD is dispersed in 100 ml of coupling buffer (0.01 M pyridine HCL, pH: 6.00, 0.1 M NaCl) through sonication for 10 minutes at high power. The suspension is then centrifuged at 14,000 rpm for 40 minutes, and the supernatant is removed after 10 minutes. After a second wash, the CQDs are suspended in a linking buffer (coupling buffer with 5% glutaraldehyde), shaken for 3 hours at 100 rpm at 25° C., and then centrifuged at 14,000 rpm for 20 minutes. Excess glutaraldehyde is removed by aspirating the supernatant and washing the CQDs with coupling buffer, as previously described. Next, 10 mg of cytarabine is dissolved in 100 ml coupling buffer and kept one ml as a precoupling buffer. The remaining solution is combined with 100 mg of washed CQDs. The mixture is shaken from end to end for 24 hours at 25° C. and then centrifuged at 8000 rpm at 4° C. to obtain standard drug NFs. The characterized conjugated CQDs are kept at −20° C. in a storage buffer solution of 0.01 M Tris-Cl, pH.

### AML Potential Analysis:

To conduct a potential analysis of AML, male Wistar rats weighing over 100 g are purchased from the animal house of the IMBB department of the University of Lahore and kept in separate cages in the animal house. All animal care and handling are performed according to our institution's guidelines for laboratory animals. The control group consisted of 4-5 weeks old males (n=3), kept in separate cages and given a normal commercial diet and distilled water.

AML is induced in the rats using N-ethyl-N-nitrosourea (ENU), which had been shown to induce leukemic effects. The AML induction protocol using intraperitoneal injection (IP) is previously reported, and all rats are kept in different cages based on their groups. The rats are fed a commercial diet and induced with ENU and NaCl at specific doses according to their weight.

After four weeks, three rats from the negative control group are sacrificed and examined to diagnose AML. The remaining rats are treated with theragnostic nanomedicines, and their weight is monitored throughout the exposure to ENU. At the time of dissection, each rat's weight is recorded, and they are anesthetized with chloroform for 10-15 minutes. The rats are then sacrificed by puncturing their hearts, and their whole blood is collected in a vial. The serum is separated and stored at −20° C. until further processing.

CBC and D-dimers are performed on the rat's whole blood, and parameters such as hemoglobin, WBCs, Total RBCs, PVC, MCV, MCH, MCHC, Neutrophils to Lymphocytes ratio (NLR), platelets, monocyte count and eosinophils are determined. LFT and RFT are performed on the thawed serum to determine alkaline phosphatase (ALP), aspartate aminotransferase (AST), alanine aminotransferase (ALT), bilirubin, albumin, total proteins, globulin, A/G ratio, creatinine, and urea.

For each slide, only one smear is made, with the dominant hand putting the edge of the second microscope slide in front of the blood drop on the first slide at an angle of about 35-45° C. The second slide is pulled back into the blood drop with gentle pressure, and the blood is allowed to spread to the edge of the slide. The top slide is moved quickly but gently through the rest of the smear to spread the blood. The smears are then dried by air and dipped in 100% methanol before being stained with a 10% Giemsa solution and distilled water. The slide is rinsed with tap water and dried with bibulous paper.

### Theragnostic Nanomedicines Effect Through Targeting Drug Delivery (In Vivo and In Vitro)

Theragnostic nanomedicines have the potential to serve as a single formulation for diagnosis, prognosis, and therapeutics. These nanomedicines have shown promising results in targeting drug delivery in vivo and in vitro.

The 24 rats of AML induction (ENU+NaCl) were used to evaluate the efficacy and drug binding to leukemia cells (in vivo and in vitro). The rats' body weight and activity are monitored before and after treatment with different nanoformulations.

The rats are divided into seven groups, with each group containing three rats. Group A is treated with saline (negative control), group B with 50 mg/kg nanocarrier in saline, group C with extract and group D with 50 mg/kg standard drug nanoformulation. Groups E to H are treated with targeted and non-targeted NFs containing 50 mg/kg CQDs in saline and injected IP three times a week.

The animals were observed for 30 days, and their activity and body weight were recorded during dose administration. The animals showed no adverse effects during the treatment, and the survival rate is effective. The results demonstrate the potential of theragnostic nanomedicines for targeted drug delivery in vivo.

After one month of treatment, the rats were given doses and dissected. Before dissection, the rats' body weight was recorded, and they were anesthetized with chloroform. A cardiac puncture is performed to sacrifice the rats, and blood was collected in three vials, including Lavender, Light-blue, and yellow caped test tubes.

20 μl of the blood is separated for fluorescent analysis, and the remaining blood is processed for various blood tests, such as CBC, LFT, RFT, D-Dimers, NLR, and blood slide study. The data obtained from these tests was analyzed using bar charts, and the results were presented as mean with standard deviations. SPSS version 26 is used for data analysis, and a result significance level of p<0.05 is declared. The graphs are constructed using Origin Pro 8.

### Theragnostic Nanomedicines Effect (In Vitro)

After the rats are dissected, the effect of theragnostic nanomedicines on targeting drug delivery is evaluated in vitro. The treated rats' blood is checked under a fluorescent microscope to assess the adhered blood cells on albumin-coated slides. The slides are fixed with methanol and then incubated at 37ºC for 20-25 minutes. After washing with PBS, the binding is visualized under a fluorescent microscope with blue and green excitation filters.

The software is used to analyze the fluorescent intensity, and the concentration of nanoparticles and targeted drugs in the blood is estimated by measuring the intensity over a 67 cm² area. These experiments provide valuable insights into the effect of theragnostic nanomedicines on targeting drug delivery in vitro.

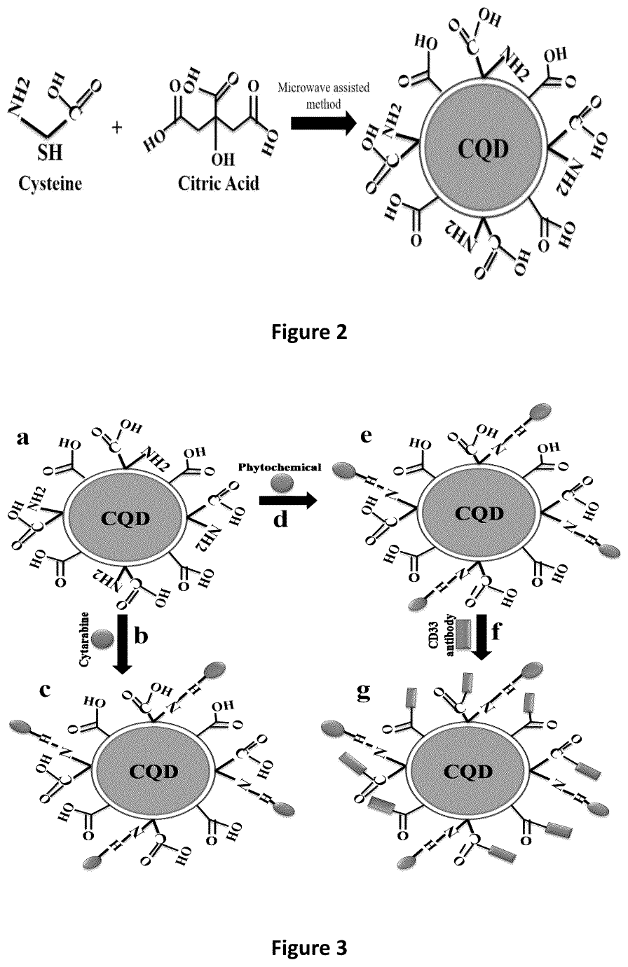

FIG. 2 illustrates a schematic presentation of the synthesis of CQD. The results of the qualitative analysis of the neem plant (A. india) are presented in Table illustrated in FIG. 21. These phytochemicals are widely used in the drug and pharmaceutical industries due to their biological and therapeutic properties. A. indica has become increasingly important because of its potential to address various health issues. Table illustrated in FIG. 21, highlighted that the aqueous extract of A. indica has the highest levels of saponin, steroid, and terpenoid, with moderate levels of tannins and low levels of phenol, alkaloids, and flavonoids.

FIG. 3 illustrates a proposed model of a reaction sequence for the synthesis of CQD theragnostic nanoformulations. Scheme demonstrating the integration of standard drug (b) on CQD (a) to make standard drug nanoformulation reacted with cytarabine and final theragnostic nanomedicine forms (c). Phytochemical (d) react with CQD (a) for reductive amination to conjugate with the amine group and activate the OH group (e). Then CD33 protein reacts with CQD (e). CD33 conjugates to the carboxylic group of CQD and final targeted theragnostic NF1 and NF2 forms (g).

FIG. 3 depicts the characterization process of theragnostic nanomedicine with CQDs. Further characterization of CQD and theragnostic nanomedicines is carried out using various techniques such as UV-Vis spectroscopy, FTIR, zeta potential, DLS, SEM, and EDX at each step.

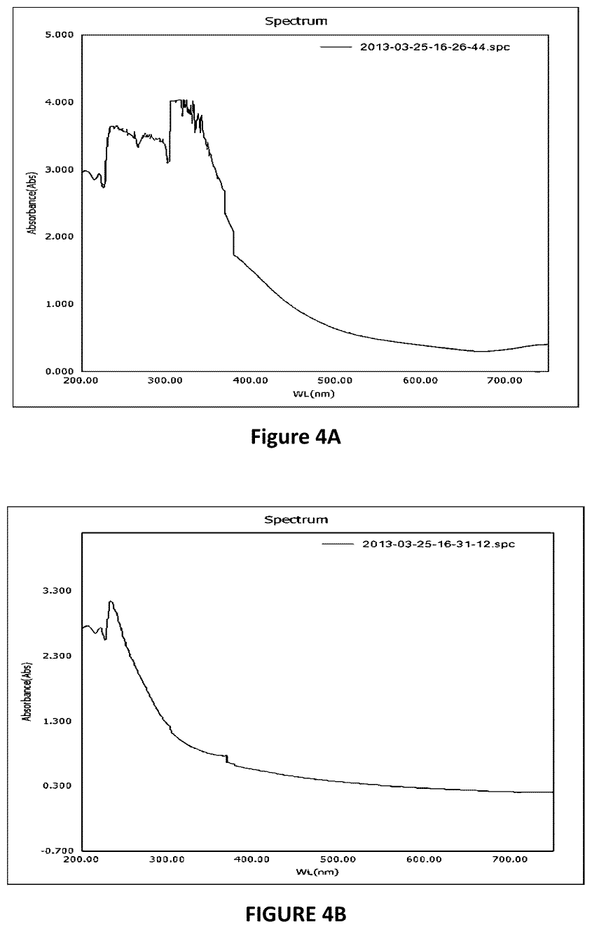

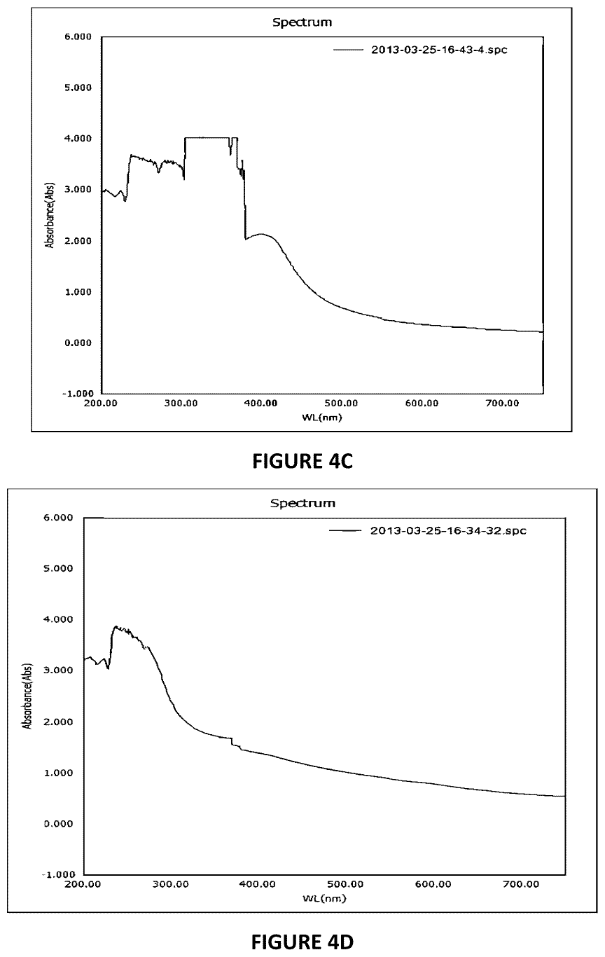

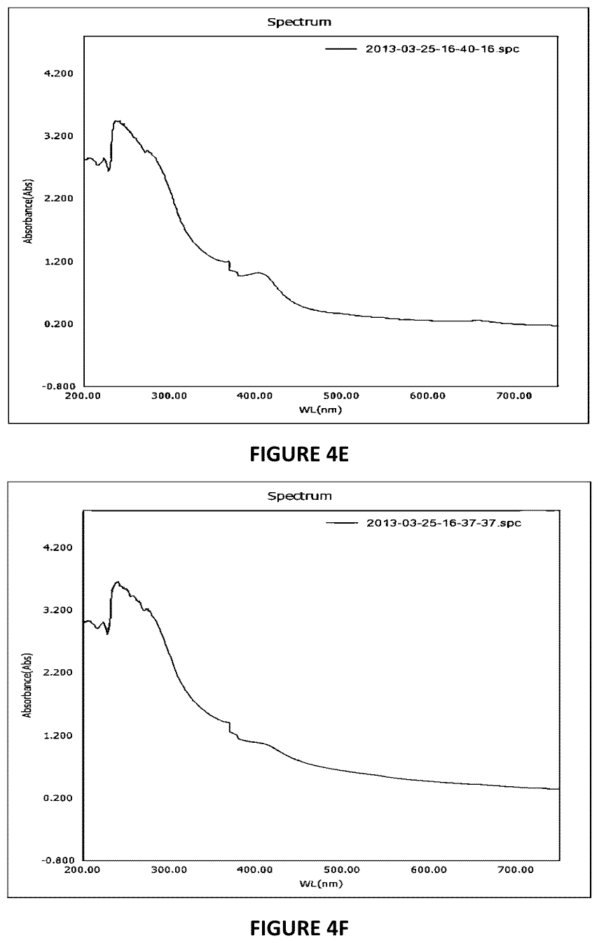

FIG. 4 illustrates the UV-Vis absorbance spectra of CQD (a). UV-Vis absorbance spectra of standard drug NF (b). UV-Vis absorbance spectra of OH-CQD (c) and NF1 (d). UV-Vis absorbance spectra of aldehyde-CQD (e) and NF2 (f). The UV-vis spectroscopy data is examined to understand better the size and size distribution of the CQDs (FIG. 4). The aqueous-soluble CQDs prepared in this study appeared yellowish-brown. They exhibited strong absorbance in the UV-blue wavelength range extending beyond 500 nm, consistent with most CQD absorption spectroscopy data. Two distinct peaks are observed at 245 and 355 nm, corresponding to the π-π* and n-π* electronic transitions of C═C/C═N and C═O double bonds. Fluorescence detection is performed under uniform conditions, with excitation wavelength and emission range set at 355 and 380-750 nm, respectively (FIG. 4a), and slit widths of the excitation and emission set at 10 nm and 15 nm, respectively (FIG. 4). UV-Vis scanning is conducted from 200 to 750 nm. For the standard drug NF, a peak is observed at 380 nm (FIG. 4b), while for the OH-CQDs, a peak is observed at 390 nm with a greater redshift (FIG. 4C). FIG. 4d shows a peak at 387 nm for NF1 and 375 nm for aldehyde-CQDs (FIG. 4e). The peak is observed at 385 for NF2, as shown in FIG. 4f. CQDs exhibited two distinct emission peaks. Different fluorescence intensities are observed for CQDs synthesized using the five different methods.

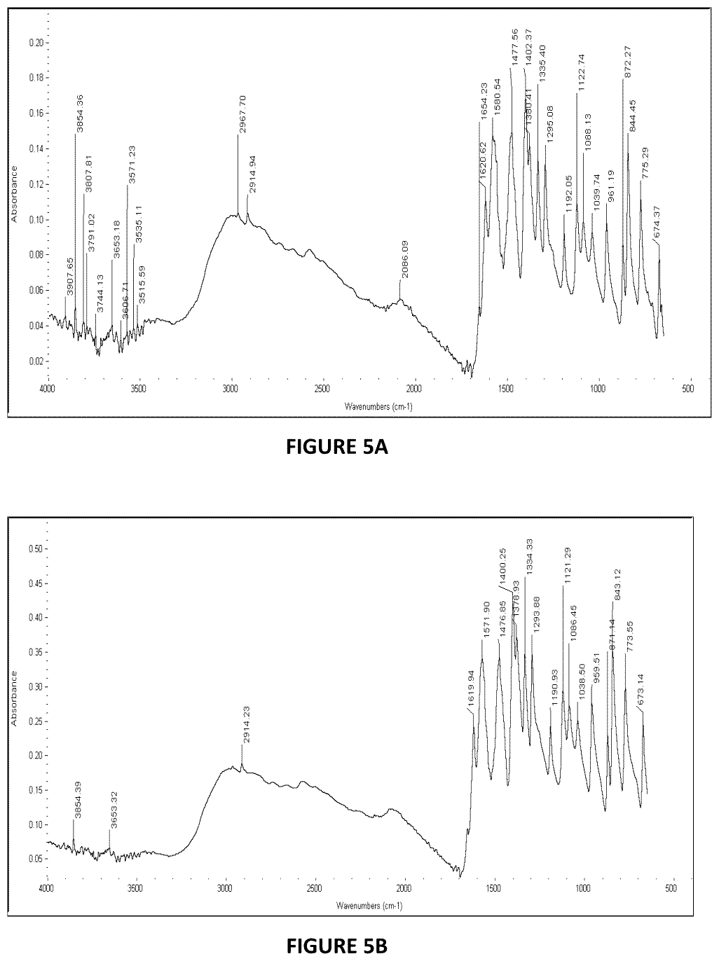

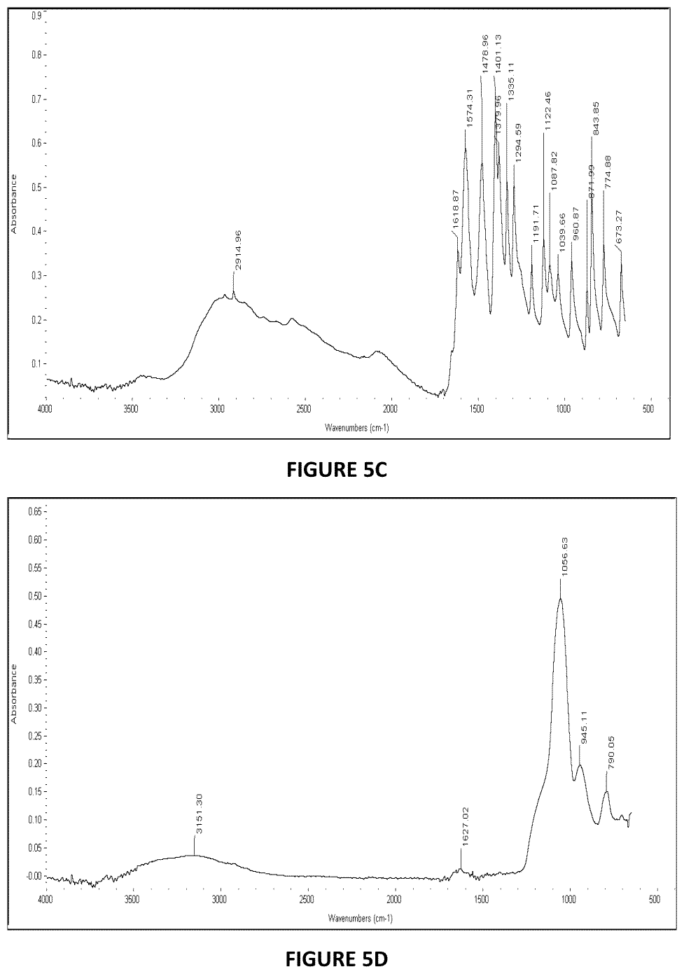

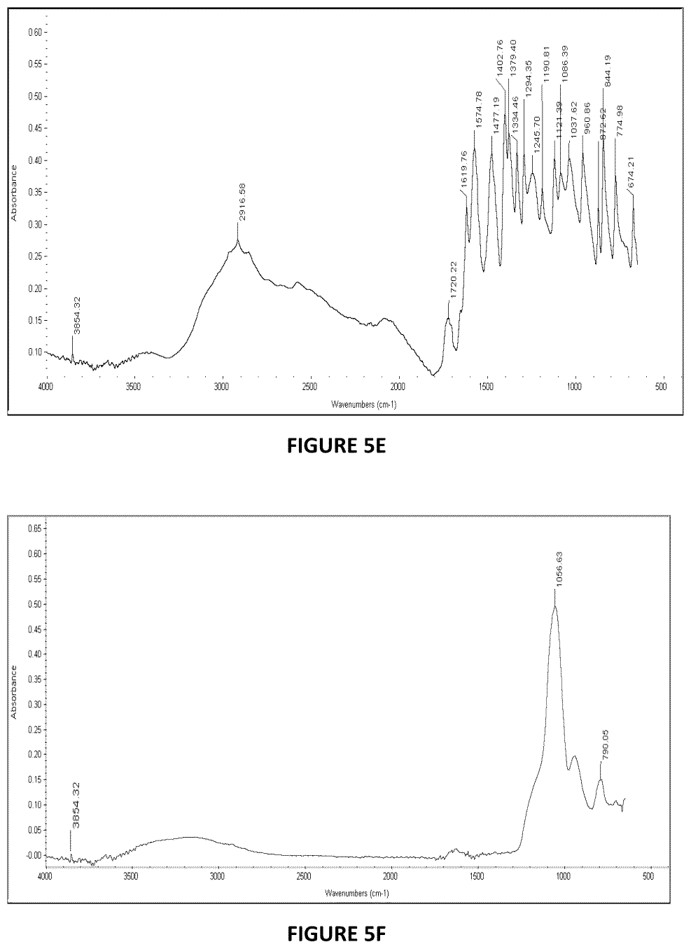

FIG. 5 illustrates the FTIR spectra of CQDs nanoparticles (a). FTIR spectra of standard drug NF (b). FTIR spectra of OH-CQDs (c) and NF1 (d). FTIR spectra of aldehyde-CQD (e) and NF2 (f). In FIG. 5, FTIR spectra revealed characteristic peaks of various functional groups present in the CQDs. The stretching vibration peaks of OH and NH are observed in the range of 3515.59-3607.81 cm⁻¹, while the minor band at 2914.94 cm⁻¹ may be attributed to the stretching vibrations of the C—H bond. The weaker peaks at 2086.09 cm⁻¹ confirmed the presence of —SH groups. The C—O bond's stretching vibration peak at 1654.23-1720.23 cm⁻¹ indicated the presence of the carboxyl group, while the peaks at 1620.62 cm⁻¹ are attributed to the stretching and bending vibrations of C—O. The C═C peak confirmed the graphitic structure of the CQDs, and the OH, C═O, and C—H bending peaks suggested the presence of corresponding surface moieties. Mild amine N—H bend peaks are seen at 1580.54 cm⁻¹, while 1380.41 cm⁻¹ is associated with the stretching vibration of C—N, N—H, and —COO bonds. FIG. 5b shows the conjugation of standard drugs with CQD. The absence of the N—H stretches of the amine group at position 3515.59-3607.81 cm⁻¹ and the bend at 3653.32 confirmed the conjugation of cytarabine with CQD nanoparticles. In FIG. 5c, a C═O peak is observed only in OH-CQDs at 1720.23 cm⁻¹, confirming the presence of the carboxyl group. The activation of the hydroxyl group of the CQDs using carbonyl-imidazole resulted in the disappearance of the OH stretch at 3515.59-3907.69 cm⁻¹ and the appearance of the C—O bond stretch at 1720.23 cm⁻¹, confirming the OH-CQD (FIG. 5c). When the aldehyde group of the CQDs is activated using cyanoborohydride and conjugated with phytochemicals, the C—O stretching peak appeared at 1720.23 cm⁻¹, and the N—H stretch of the amine group at 3854.32 cm⁻¹ disappeared (FIG. 5e). Carbodiimide is used to activate the carboxyl group present on both OH-CQDs and aldehyde-CQDs during the conjugation of phytochemicals with CQDs. The O—H stretching at 3515.59-3907.69 cm-1 disappeared in both phytochemical conjugated CQDs, confirming the conjugation of protein on CQDs (FIG. 5d, 5f).

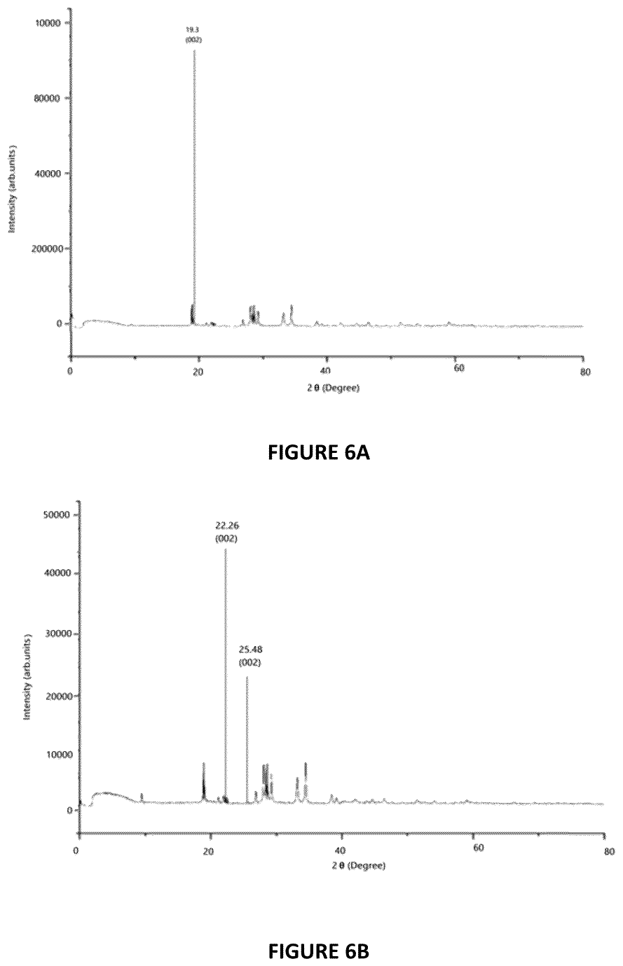

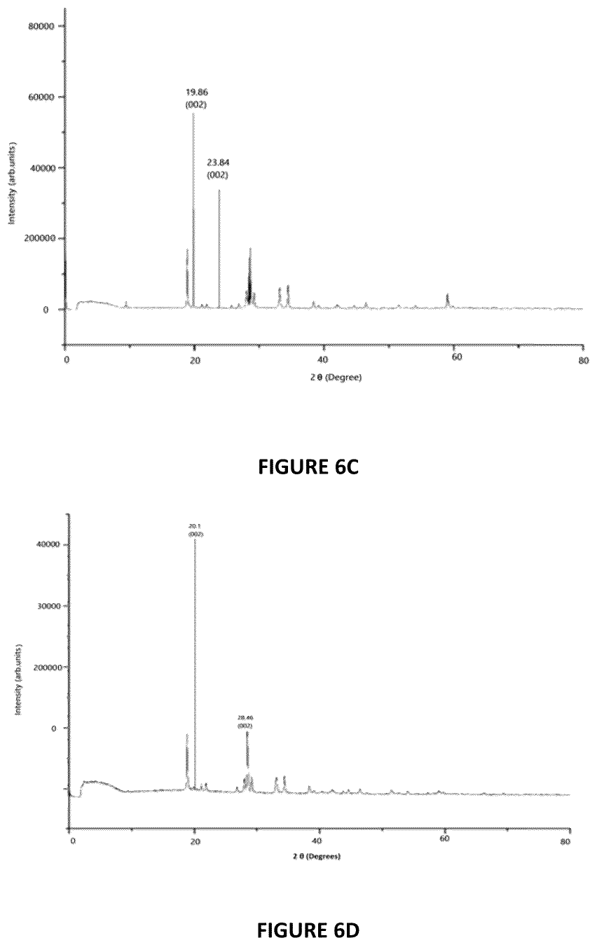

FIG. 6 illustrates the XRD of CQD (FIG. 6 a). XRD of standard drug NF (FIG. 6b). XRD of NF1 (FIG. 6c) and NF2 (FIG. 6d). In FIG. 6, the characterization of the CQDs is carried out using X-ray diffraction (XRD). The presence of graphitic carbon results in a clear diffraction peak in the 20 range of 19-24°, corresponding to the (002) plane. The CQDs' lattice spacing, approximately 0.402 nm, is determined based on the Bragg formula (1.1). These findings indicate that the CQDs have amorphous structures with a graphitic carbon core that includes sp2 hybrid carbon.

$$2 d sin θ = n λ \qquad ( 1.1 )$$

Where d is the CQD lattice spacing, θ is the diffraction angle, n is the reflection series, and λ is the wavelength.

The X-ray diffraction (XRD) analysis of CQDs is presented in FIG. 6a. The peak at around 19.3ºC indicates that the CQDs are naturally occurring amorphous carbon particles, as determined by XRD. The standard drug nanoformulation's XRD pattern in FIG. 6b shows a large peak at 22.26° ° C. and a small peak at 25.48° C., corresponding to (002). FIG. 6c displays a large peak at 19.86° C. and a small peak at 23.84° C. corresponding to (002) in the XRD pattern of the NF1. In FIG. 6d, the XRD pattern of the NF2 shows a large peak at 20.1° C. and a small peak at 28.46° C., corresponding to (002).

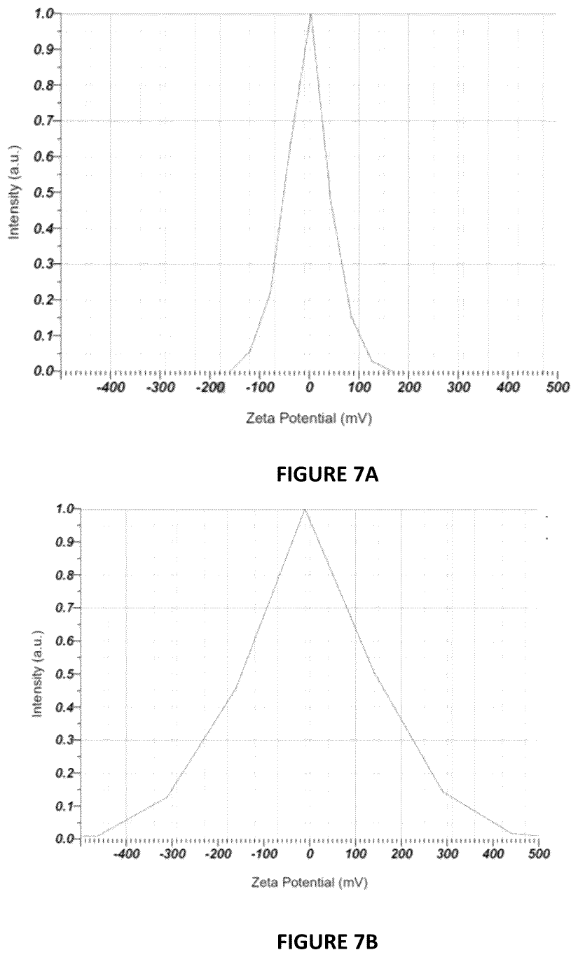

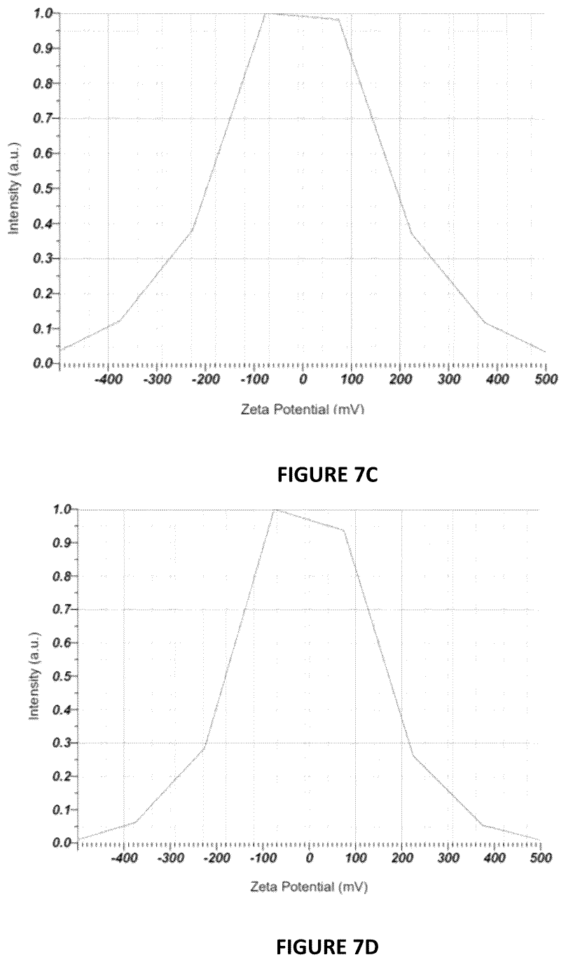

FIG. 7 illustrates the Zeta potential of CQD nanoparticles (a). Zeta potential of standard drug NF (b). Zeta potential of targeted NF1 (c). Zeta potential of targeted NF2 (d). In FIG. 7, the size distribution of as-synthesized CQDs is confirmed by a zeta-sizer. The zeta-potential of CQDs is found to have a single peak at −3.1 Mv (FIG. 7a), suggesting negative charge moieties on the CQD surface. The high concentration of hydroxyl, carbonyl, and carboxyl groups on the CQD surface in aqueous media resulted in a zeta potential of −3.1 Mv, indicating the negatively charged CQDs' high dispersion and stability in aqueous solutions. FIG. 7b shows that even after conjugation, the zeta potential of standard drug nanoformulation remained negative at −3.8 Mv, indicating the continued presence of carboxyl groups on the CQD surface. FIG. 7c showed NF1 zeta potential to −3.3 Mv, indicating a tendency to form bonds on the surface of CQD nanoparticles. FIG. 7d showed NF2 zeta potential of −5.4 Mv, indicating an even stronger tendency to form bonds on the surface of CQD nanoparticles.

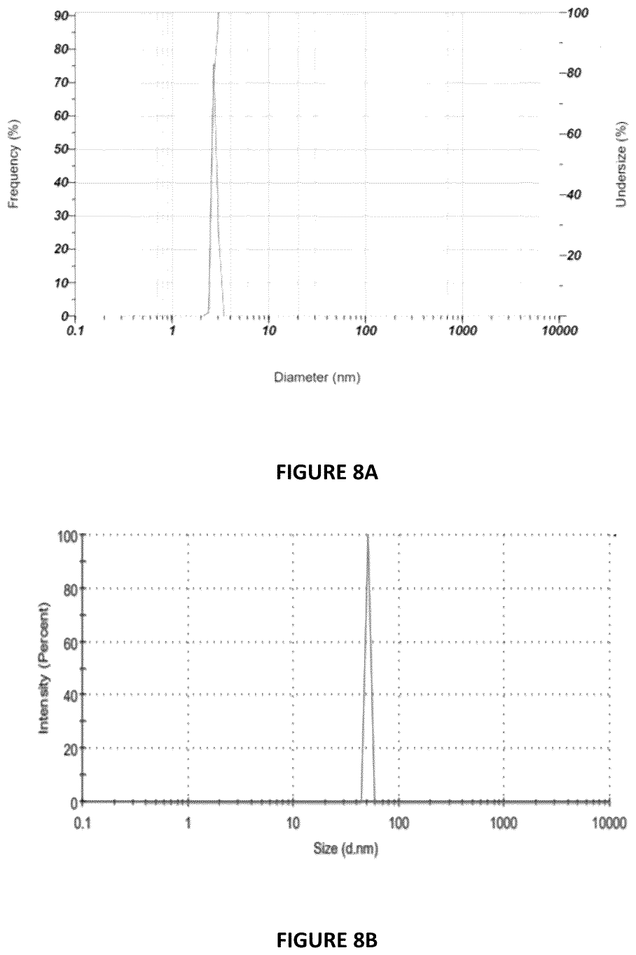

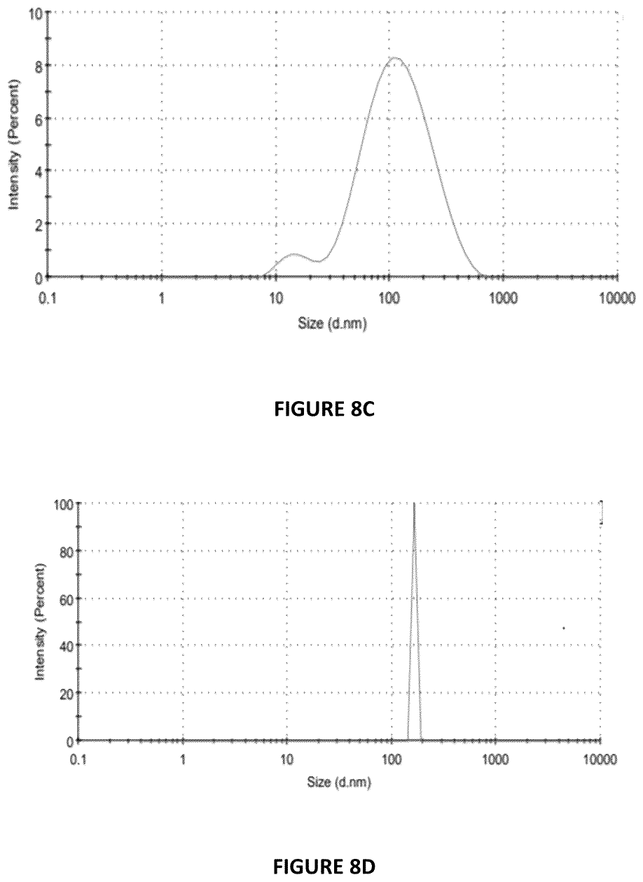

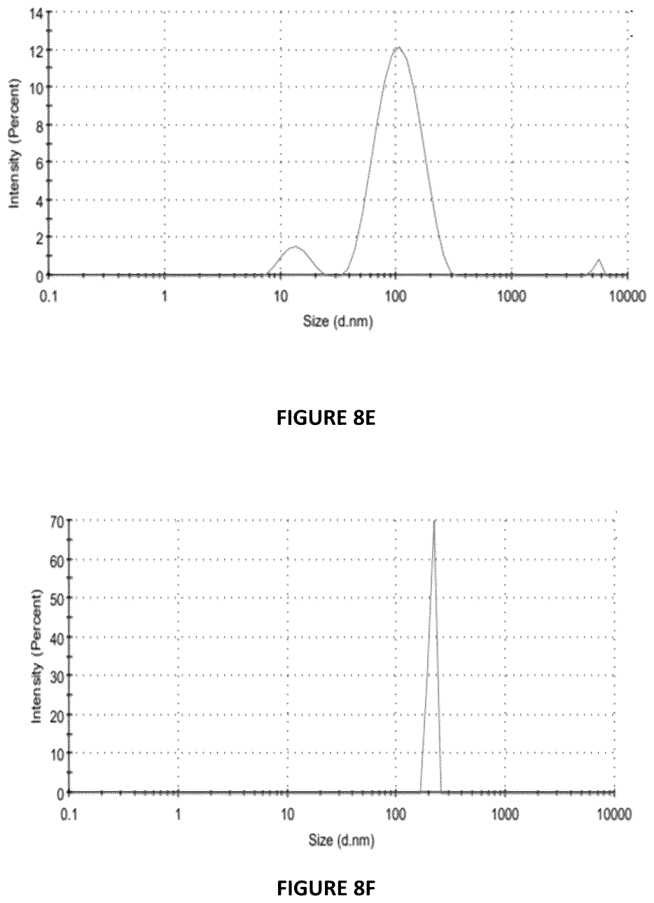

FIG. 8 illustrates the DLS analysis of CQD nanoparticles (a). DLS analysis of standard drug NF (b). DLS analysis of OH-CQD (c) and NF1 (d). DLS analysis of aldehyde-CQD (e) and NF2 (f). FIG. 8 displays the size and dispersion of CQD nanoparticles analyzed by DLS. The size of CQD nanoparticles ranged from 2-3.3 nm, with an average size of 2.909 nm (FIG. 8a). DLS analysis of standard drug nanoformulation showed a distribution of nanoparticles with a mean size of 50 nm and a size range of less than 100 nm (FIG. 8b). The size of OH-CQD nanoparticles ranged from 15 nm to 100 nm and 100 nm to 600 nm, with an average size of 140 nm (FIG. 8c). Similarly, after NF1, the size range of nanoparticles is reported to be between 150 to 200 nm, with an average size of 164 nm (FIG. 8d). The size of aldehyde-CQD nanoparticles ranged from 13 nm to 23 nm and 40 nm to 300 nm, with a mean size of 112 nm (FIG. 8e). Some of the aldehyde-CQD nanoparticles showed a larger size due to phytochemical conjugation. Following protein attachment to the NF2, a size range of 150 to 250 nm is observed with a mean size of 211 nm, suggesting that the nanoparticles' size became stable (FIG. 8f).

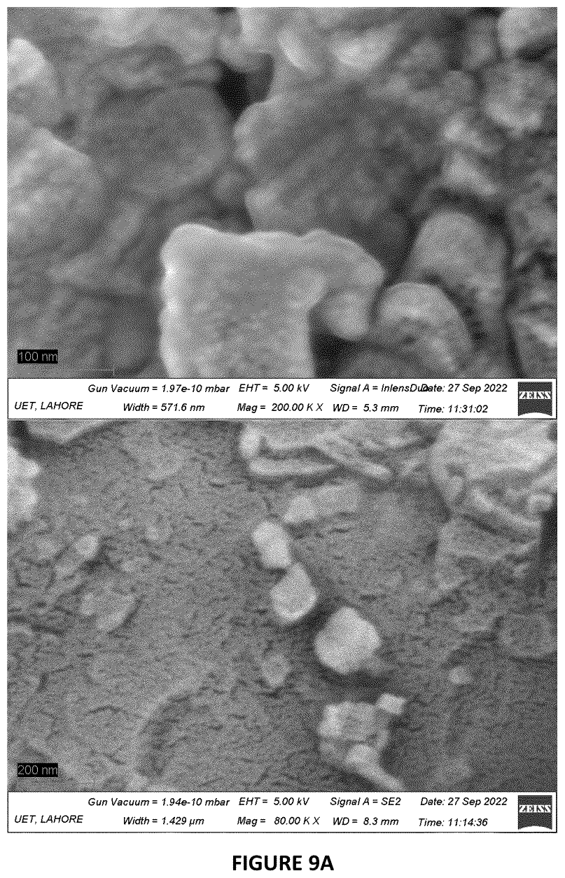

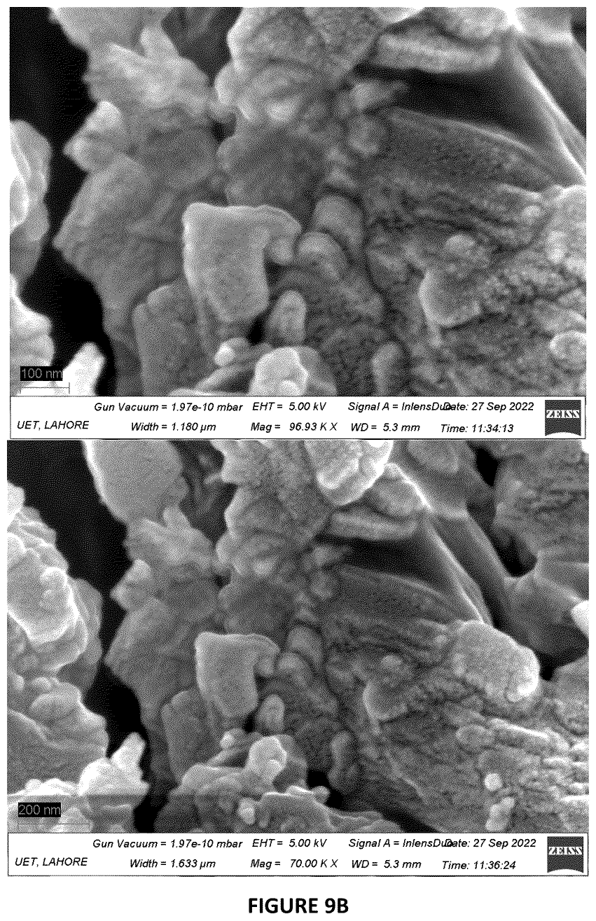

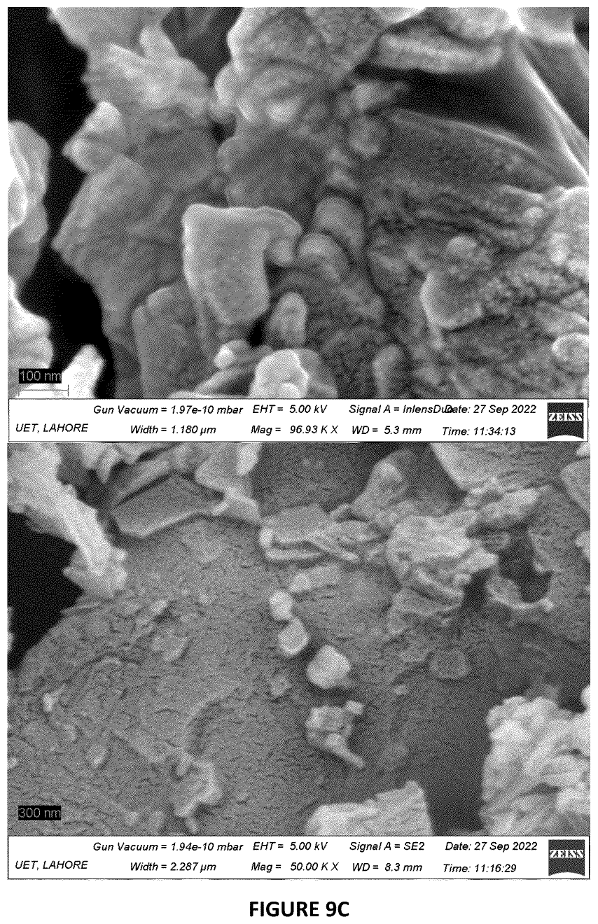

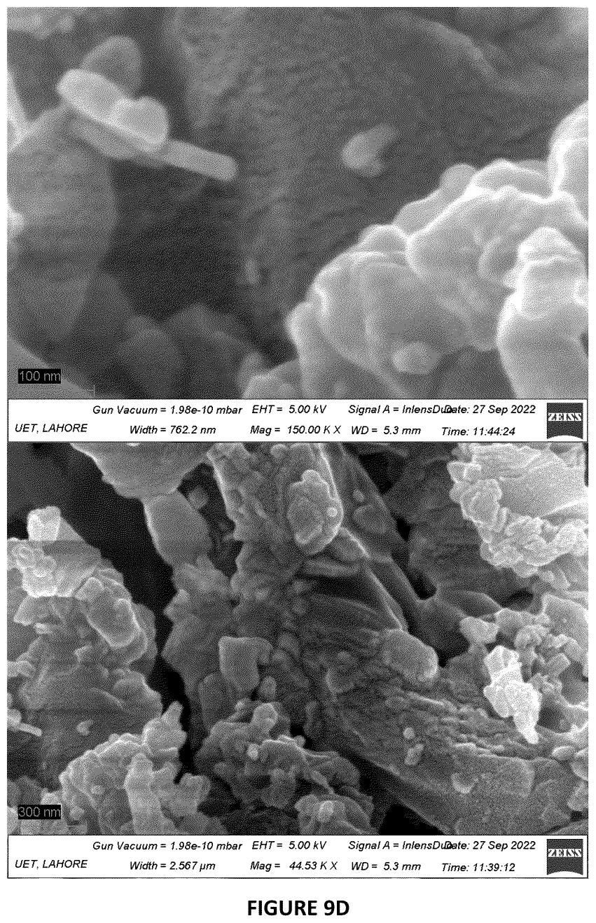

FIG. 9 illustrates a) SEM images of CQDs at 200 nm and 100 nm scales, b) SEM images of standard drug NF at 200 nm and 100 nm scales, c) SEM images of targeted NF1 at 300 nm and 100 nm scales, and d) SEM images of targeted NF2 at 300 nm and 100 nm scales. FIG. 9 shows that the SEM equipped with EDX is used to demonstrate the synthesized nanomedicine's morphological features and elemental composition. Different magnifications of 100 nm and 200 nm are used to capture the surface morphology of CQDs. FIG. 9a depicts irregular spherical shapes of CQDs, with a diameter of around 10-40 nm. The high-energy electron beam used in SEM provides information about the solid specimen, resulting in signals focused on the targeted specimen. The standard drug nanoformulation showed an irregular cubic morphology with a diameter of approximately 50-110 nm, as seen in FIG. 9b. The targeted NF1 and NF2 exhibited aggregated particles of various sizes, with a diameter of less than 100 nm due to phytochemicals, as depicted in FIG. 9c and FIG. 9d.

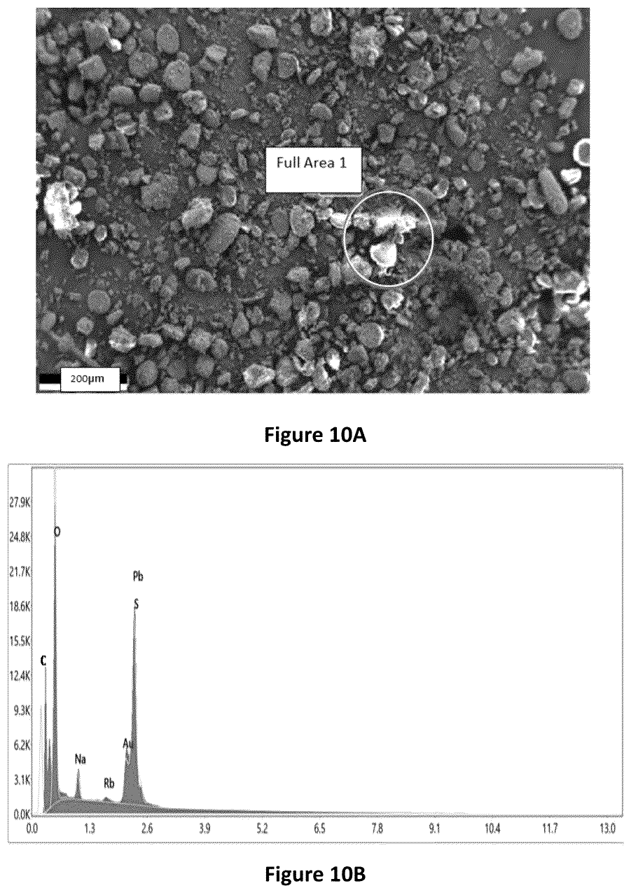

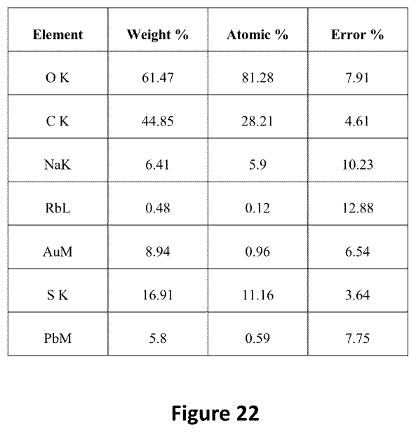

FIG. 10 illustrates a) EDX image of elemental composition of the CQDs and b) EDX micrograph of CQDs. To determine the elemental composition of the synthesized CQDs, EDX analysis is carried out, as shown in FIG. 10a. Table illustrated in FIG. 22 shows that the EDX confirmed the presence of O, C, and other elements. The EDX spectrum of the CQDs revealed peaks corresponding to Oxygen (O), Carbon (C) and Sodium (Na) with atomic percentages of 81.28%, 44.85%, and 6.41%, respectively. Other elements present in the CQDs with significant weight % are Rubidium (Rb), Gold (Au), Sulfur (S), and Lead (Pb). CQDs only showed C and O signals. The EDX data confirmed that the CQDs maintained their morphology while being functionalized with C and O. The elemental composition of the CQDs is highlighted in the EDX image, covering the entire area of the figure, as shown in FIG. 10b. The micrograph depicted the elements O, C, Na, Rb, Au, S, and Pb, with atomic and weight percentages of O, C, Na, Rb, Au, S, and Pb being 81.28, 28.21, 5.9, 0.12, 0.96, 11.16, and 0.59, and 61.47, 44.85, 6.41, 0.48, 8.94, 16.91, and 5.8, respectively.

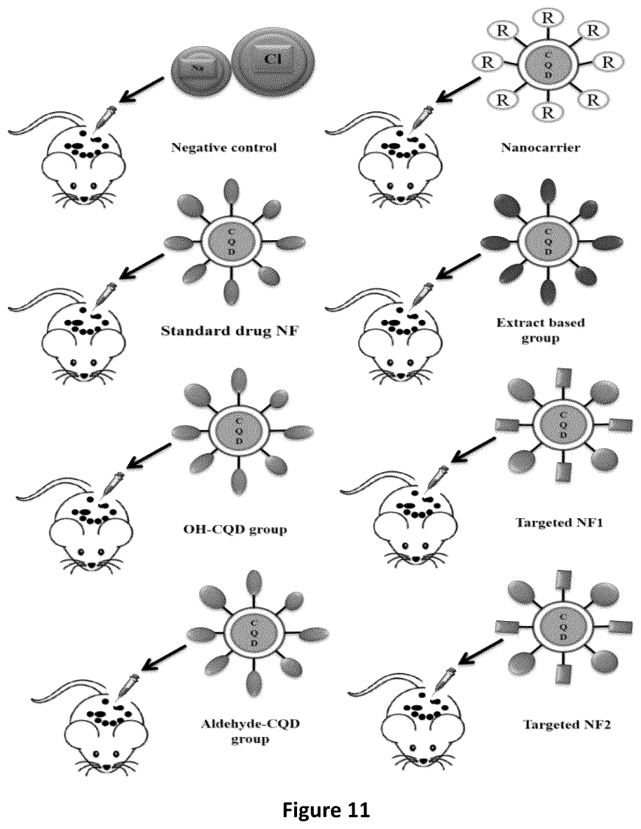

FIG. 11 illustrates the schematic presentation of injected theragnostic nanoformulations group in rats. The monitoring of treatments for 30 days is presented in FIG. 11.

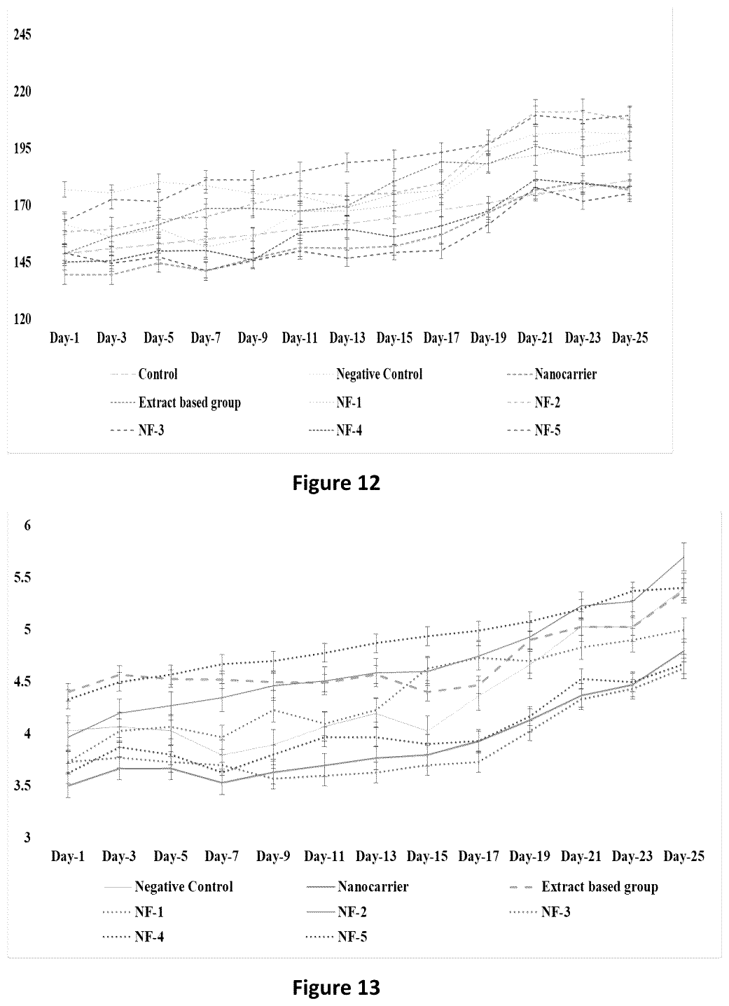

FIG. 12 illustrates the weight of rats (Induction) from Day-1 till Day-25. The weight of AML induction decreased daily after inducing ENU doses (FIG. 12).

FIG. 13 illustrates the Doses of rats (Induction) from Day-1 to till Day-23. The doses of ENU increased as the weight decreased (FIG. 13).

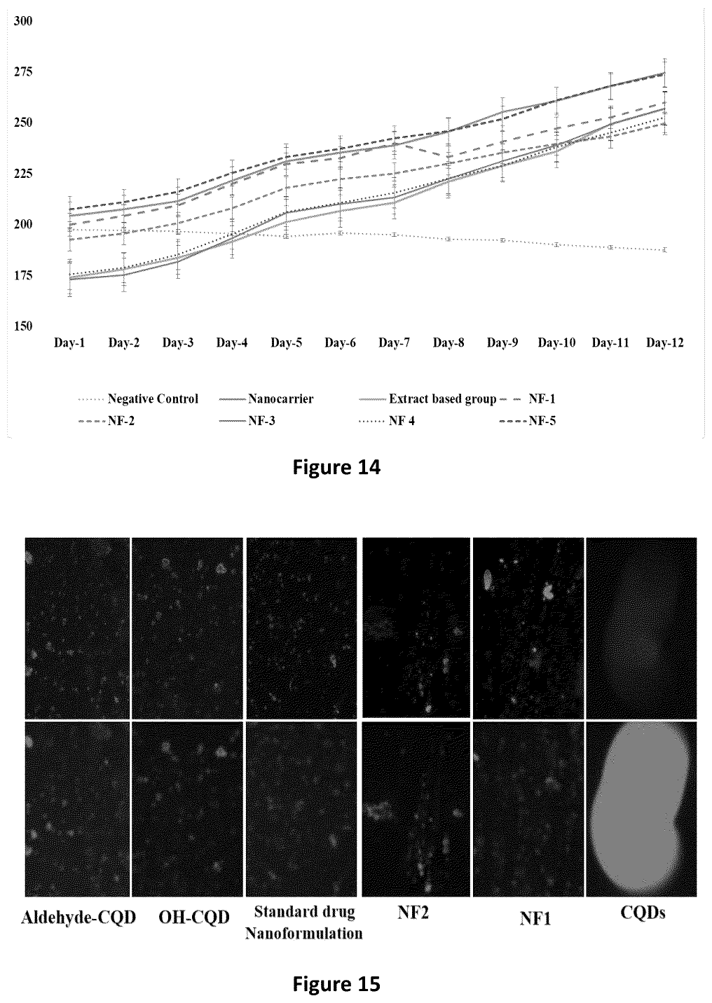

FIG. 14 illustrates the weight of rats (Therapeutic) from Day1 till Day 12. The nanoformulations are active in treated rats, although some rats experienced dizziness after dosing, which subsided quickly, and they resumed regular activity. The impact of the treatment on weight gain or loss for 30 days is summarized in FIG. 14. All rats treated with CQD theragnostic nanomedicine lived longer and usually gained weight.

FIG. 15 illustrates In vitro receptor binding of CQD theragnostic nanomedicine on leukemia blood slides. Different fluorescent filters indicate the presence of CQD nanoparticles and nanoformulations in the blood. Fluorescence only in blood showed blood-only receptor targeting and binding of nanoformulations.

In vitro investigations verified that the formulation of theragnostic nanomedicine impacts the binding site of AML receptors. FIG. 15 demonstrates the successful attachment of CQD theragnostic nanomedicine to AML blood receptors. The presence of CQD nanoparticles in the blood is observed through blue and green fluorescent filters. The NF1 and NF2 bind to the AML cell receptors effectively.

Effect of CQD theragnostic nanomedicine on leukemia treatment: Control, negative control group, and therapeutics group (nanocarrier, extract, standard drug, OH-CQD, Aldehyde-CQD, NF1, and NF2) are examined through blood tests (CBC, LFT, RFT, and D-Dimers). Standard drug nanoformulation, NF1 and NF2, showed the best results.

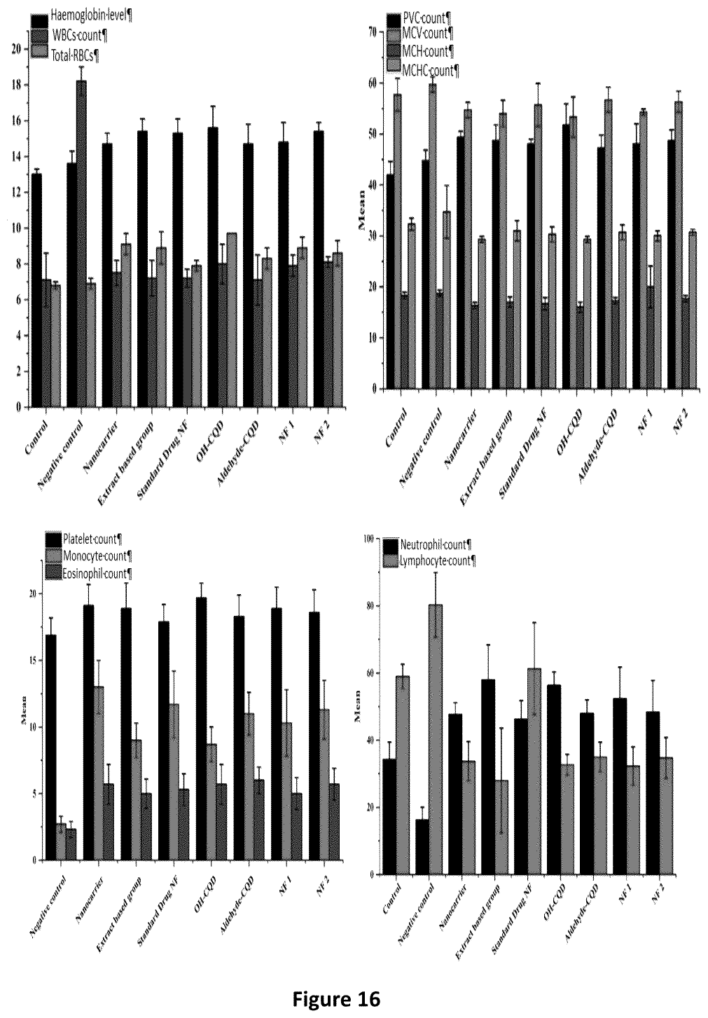

FIG. 16 illustrates the complete blood count levels in control and treatment groups.

### Level of Biochemical Markers in Control, Negative Control, and Treatment Groups:

The study compared the effects of various nanomedicines on blood parameters in a treatment group and compared them to control and negative control groups, as shown in FIG. 16. The mean WBCs level of the treatment groups is significantly near to the control (p<0.047). The effective nanomedicines are Aldehyde-CQD, standard drug nanoformulation, extract, NF1 and NF2 groups than others treatment groups. The treatment group had significantly higher mean haemoglobin levels, total RBCs, and PVC count than the control and negative control groups. Aldehyde-CQD and standard drug nanoformulation are the most effective nanomedicines. The mean MCV count is similar to the control, with CQD-Aldehyde, standard drug nanoformulation, NF1, and NF2 being the most effective. The mean platelet count is similar to control and negative control, with nanocarrier and NF1 being the most effective nanomedicines. Neutrophil and lymphocyte levels are similar to control and negative control, with NF1 and NF2 being the most effective nanomedicines. The mean monocyte count and eosinophil count are higher in the treatment group than control and negative control, with OH-CQD, extract-based group, NF1, and NF2 being the most effective nanomedicines.

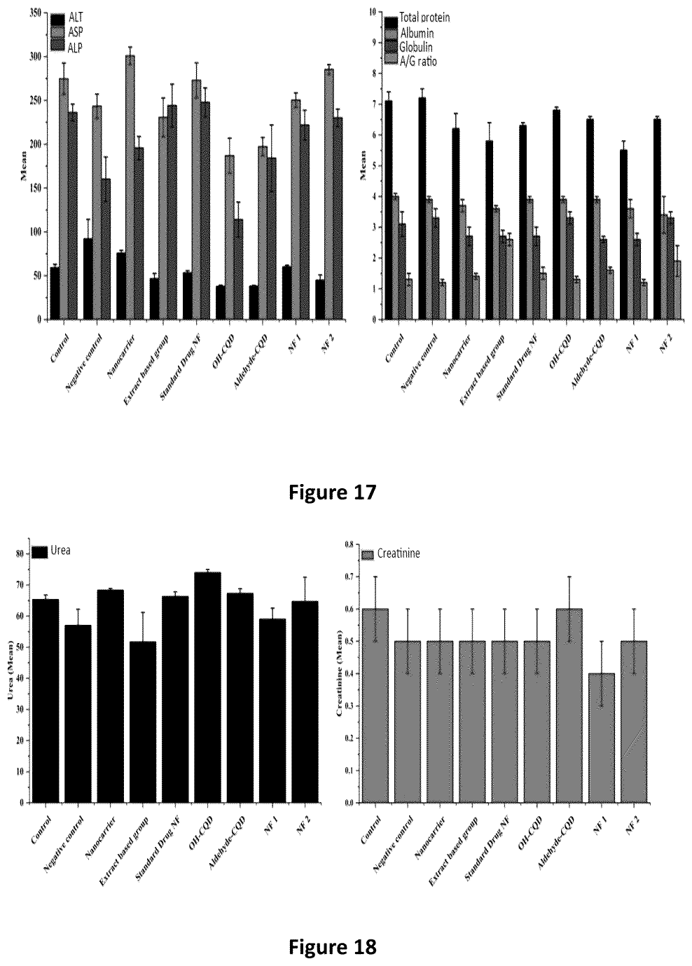

FIG. 17 illustrates the LFT Parameters in control and treatment groups. In FIG. 17, the negative control group had high ALT, while AST and ALP levels are low in both groups. The effectiveness of theragnostic medicines is demonstrated as all treatments are closer to the control group, with standard drug nanoformulation, NF1, and NF2 showing the best results.

FIG. 18 illustrates the RFT Profile in control and treatment groups. In FIG. 18, total protein, albumin, and globulin are higher in the negative control group than in the control group, while albumin is lower in these groups. Urea and creatinine levels are higher in the negative control compared to the control group. All treatments are closer to the control group, demonstrating the effectiveness of theragnostic medicines. Overall, all nanomedicines showed the best results in the RFT profile.

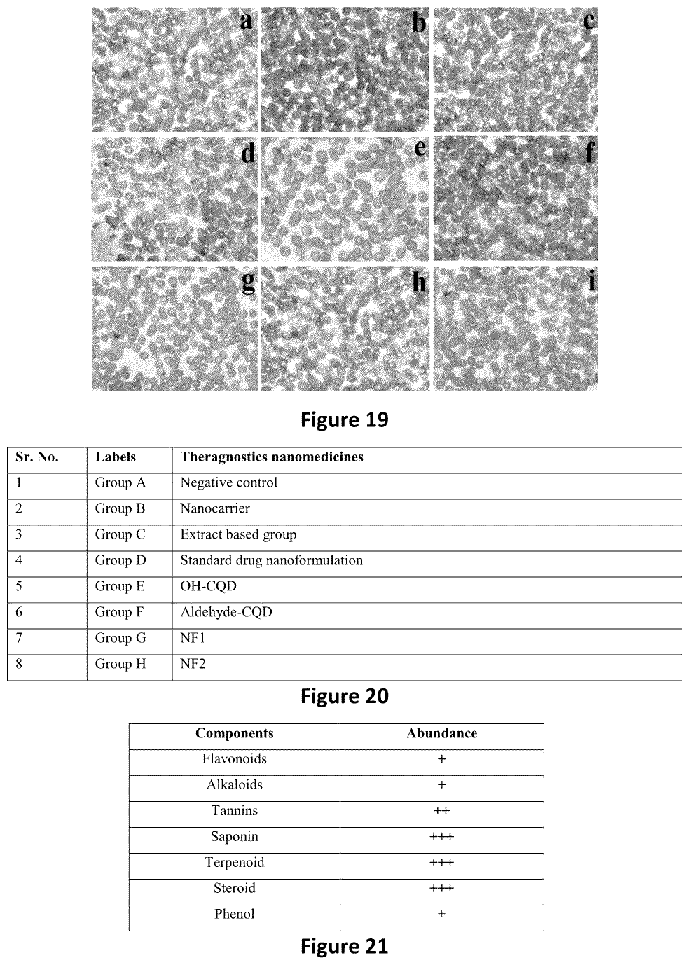

FIG. 19 illustrates the Macroscopic examination of rat blood treated with theragnostic nanoformulations. (a) Control group (b) Negative control (c) Nanocarrier (d) Extract based group (e) Standard drug nanoformulation (f) OH-CQD group (g) Aldehyde-CQD (h) NF1 (i) NF2. FIG. 19 shows histological sections of all rats in the study group, which demonstrated the presence of AML. In healthy rats (FIG. 19a), blood cells, especially WBCs and RBCs, are in the normal range. On the other hand, the negative control group (FIG. 19b) had high levels of abnormal WBCs and fewer neutrophils. However, all therapeutic groups showed fewer abnormal WBCs and an increased number of neutrophils, significantly better than the negative control group.

FIG. 20 illustrates a Table depicting labeled treated groups of theragnostic nanomedicines.

FIG. 21 illustrates a Table depicting the phytochemical content of neem plant (A. india).

FIG. 22 illustrates a Table depicting the elemental composition of the CQDs.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above about specific embodiments. However, the benefits, advantages, solutions to problems, and any component(s) that may cause any benefit, advantage, or solution to occur or become more pronounced are not to be construed as a critical, required, or essential feature or component of any or all the claims.

1. A method for preparing theragnostic carbon quantum dot nanomedicine composition, the method comprises:
collecting and processing fresh plant samples of Azadirachta indica;
mixing 50 g of crushed dried neem leaves powder with 500 mL of methanol and left to extract for 1 week;
filtering the mixture and condensing using a rotary evaporator, and lyophilizing to obtain the extract;
dissolving 0.57 g L-cysteine in 10 mL of deionized water and mixing with 1.5 g of citric acid to form a solution;
ultrasonicating the solution for 10 minutes and then heating in a microwave oven for 4 minutes;
cooling the solution to room temperature and then dissolving in 10 mL of deionized water to obtain a brown solution;
centrifuging the brown solution at 9000 rpm for 10 minutes to remove impurities and filtering with a filter;
extracting the Carbon quantum dot (CQD) solution with ethyl acetate and storing in a refrigerator at 4° C.; and
synthesizing phenolic conjugated carbon quantum dots (CQDs) with hydroxyl groups (OH-CQDs), CD33 conjugated phenol-CQDs (CD33-P-CQDs), aldehyde conjugated carbon quantum dots (Aldehyde-CQDs), CD33 conjugated aldehyde-CQDs (CD33-A-CQDs), and standard drug nanoformulation (NF) comprising cytarabine conjugated to carbon quantum dots (CQDs) using the glutaraldehyde method.

2. The method of claim 1, wherein synthesizing phenolic conjugated carbon quantum dots (CQDs) with hydroxyl groups (OH-CQDs), comprising the steps of:
conjugating particles with 4.28 μg/μl of CQDs and a hydroxyl group-containing phytochemical;
suspending lyophilized methanol extracts in a Tetrahydrofuran (THF) suspension containing N, N′-carbonyldiimidazole (CDI) at 50 mg/ml and mixing for 2 hours at room temperature;
washing activated particles with THE to remove excess CDI and byproducts;
resuspending the particles at 10 mg/ml in a cold coupling buffer and washing with ice-cold deionized water, wherein the cold coupling buffer containing 0.1M sodium carbonate at 9.5 pH; and
adding amine-containing nanoparticles (NPs) and allowing them to react by mixing for at least 18 hours at 4ºC.

3. The method of claim 2, wherein the particles are washed with ice-cold deionized water after resuspension.

4. The method of claim 1, wherein synthesizing CD33 conjugated phenol-CQDs (CD33-P-CQDs), comprising the steps of:
taking 10 mg of phenolic-conjugated CQDs (OH-CQDs) in 10 ml of coupling buffer adjusted to pH 8 with 1 M NaOH, wherein the coupling buffer comprises 50 mM sodium phosphate;
combining 50 μL of rabbit polyclonal CD33 in 10 ml of coupling buffer with the OH-CQDs solution, followed by thorough mixing;
adding 10 mg of 1-ethyl-3-[3-dimethylaminopropyl] carbodiimide (EDC) in a 1:1 ratio with CQDs to the solution to activate the carboxylic groups of the CQDs, while maintaining pH at 6.4 with 1 M HCl;
incubating a reaction mixture for 2 hours at 37 ºC in a dark with continuous shaking at 100 rpm;
removing unattached molecules by centrifugation at 12,000 rpm for 10 minutes at 40° C., followed by washing three times with TBS, wherein 20 ml TBS is used for each time wash; and
suspending the washed particles in TBS buffer and storing them at −20° C. for later use.

5. The method of claim 1, wherein synthesizing aldehyde conjugated carbon quantum dots (Aldehyde-CQDs), comprising the steps of:
conjugating plant phytochemicals to amine-functionalized CQDs, wherein the plant phytochemicals are oxidized and suspended in 0.05M sodium carbonate and 0.1M sodium citrate at pH=9.6 and 1 mg mL-1;
adding amine-functionalized CQDs to the phytochemicals with an aldehyde group, dissolved in a buffer with a concentration of 1 to 10 mg/ml at pH 8-10, with a molar ratio of 1:4;
adding 10 μL of 5M-sodium cyanoborohydride in IN NaOH per mL of conjugation solution and allowing the solution to react for 2 hours on a rotator;
blocking unreacted sites by adding 10 μL ethanolamine, pH=9.6 per mL of the conjugation solution volume, and allowing it to react for 30 minutes on a rotator; and
purifying the particles through dialysis for 15 minutes and storing the purified particles at 4ºC.

6. The method of claim 1, wherein synthesizing CD33 conjugated aldehyde-CQDs (CD33-A-CQDs), comprising the steps of:
taking 10 mg of aldehyde-CQDs and 50 μL of CD33 in 10 ml of coupling buffer at pH 8 with 1 M NaOH, wherein the coupling buffer containing 50 mM sodium phosphate;
mixing the solutions thoroughly and adding 10 mg of 1-ethyl-3-[3-dimethylaminopropyl] carbodiimide (EDC) in a 1:1 ratio with CQDs to activate the carboxylic groups at pH 6.4 with 1 M HCl;
incubating the reaction mixture for 2 hours at 37° C. in the dark with continuous shaking at 100 rpm;
centrifuging the solution at 12,000 rpm for 10 minutes at 4° C. to remove unattached molecules and washing the particles three times with 20 ml each time TBS; and
suspending the washed particles in 1×TBS buffer and storing the suspended particles at −20° C.

7. The method of claim 1, wherein preparing a standard drug nanoformulation (NF) comprising cytarabine conjugated to carbon quantum dots (CQDs) using the glutaraldehyde method, comprising the steps of:
dispersing 100 mg of CQDs in 100 ml of coupling buffer through sonication for 10 minutes at high power, wherein the buffer has 0.01 M pyridine HCl, pH: 6.00, 0.1 M NaCl;
centrifuging the CQD suspension at 14,000 rpm for 40 minutes and removing the supernatant after 10 minutes;
washing the CQDs with coupling buffer as described, and suspending them in a linking buffer, wherein the linking buffer contains coupling buffer with 5% glutaraldehyde;
shaking the CQDs in the linking buffer for 3 hours at 100 rpm at 25° C. and then centrifuging at 14,000 rpm for 20 minutes;
removing excess glutaraldehyde by aspirating the supernatant and washing the CQDs with coupling buffer as previously described;
dissolving 10 mg of cytarabine in 100 ml coupling buffer, reserving one ml as a pre-coupling buffer, and combining the remaining cytarabine solution with 100 mg of washed CQDs;
shaking the mixture from end to end for 24 hours at 25° C. and centrifuging the mixture at 8000 rpm at 4° C. to obtain standard drug nanoformulations (NFs); and
keeping the characterized conjugated CQDs at −20° C. in a storage buffer solution of 0.01 M Tris-Cl, pH.

8. The method of claim 9, wherein the CQDs are dispersed in the coupling buffer through sonication for 10 minutes at high power.

9. A theragnostic carbon quantum dot nanomedicine composition prepared using method of claim 1, the composition comprises:
a powder extract of Azadirachta indica, from 40-60 grams in 400-600 mL of methanol;
a powder extract of L-cysteine, from 0.3-1 grams in 5-15 mL of deionized water;
an aqueous extract of citric acid, from 1-2 grams; and
an aqueous extract of ethyl acetate, from 0-10 mL.

10. The composition of claim 9, wherein the weight amount of Azadirachta indica, L-cysteine, citric acid, methanol, and deionized water is 50 g, 0.57 g, 1.5 g, 500 mL, and 10 mL, respectively.
